SEQUENCE LISTING (14 sequences)

SEQ ID NO: 1 - DNA, 20 bp, Homo sapiens
  source 1..20: mol_type = genomic DNA
ctctcggatctccaaattta

SEQ ID NO: 2 - DNA, 20 bp, Homo sapiens
  source 1..20: mol_type = genomic DNA
catcatagctaccatttatt

SEQ ID NO: 3 - RNA, 21 nt, synthetic construct
  source 1..21: mol_type = other RNA
taaatttggagatccgagaga

SEQ ID NO: 4 - RNA, 16 nt, synthetic construct
  source 1..16: mol_type = other RNA
cggatctccaaattta

SEQ ID NO: 5 - RNA, 21 nt, synthetic construct
  source 1..21: mol_type = other RNA
tataaatggtagctatgatga

SEQ ID NO: 6 - RNA, 16 nt, synthetic construct
  source 1..16: mol_type = other RNA
atagctaccatttata

SEQ ID NO: 7 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = OTHER; note = 2'-O-methyl-adenosine
  modified_base 4: mod_base = OTHER; note = 2'-Fluoro-adenosine
  modified_base 5: mod_base = OTHER; note = 2'-Fluoro-uridine
  modified_base 6: mod_base = OTHER; note = 2'-Fluoro-uridine
  modified_base 7: mod_base = um
  modified_base 8: mod_base = OTHER; note = 2'-Fluoro-guanosine
  modified_base 9: mod_base = gm
  modified_base 10: mod_base = OTHER; note = 2'-Fluoro-adenosine
  modified_base 11: mod_base = gm
  modified_base 12: mod_base = OTHER; note = 2'-Fluoro-adenosine
  modified_base 13: mod_base = um
  modified_base 14..21: mod_base = OTHER; note = linked via phosphorothioate
  modified_base 14: mod_base = OTHER; note = 2'-Fluoro-cytidine
  modified_base 15: mod_base = cm
  modified_base 16: mod_base = OTHER; note = 2'-Fluoro-guanosine
  modified_base 17: mod_base = OTHER; note = 2'-O-methyl-adenosine
  modified_base 18: mod_base = gm
  modified_base 19: mod_base = OTHER; note = 2'-O-methyl-adenosine
  modified_base 20: mod_base = OTHER; note = 2'-Fluoro-guanosine
  modified_base 21: mod_base = OTHER; note = 2'-O-methyl-adenosine
  source 1..21: mol_type = other RNA
taaatttggagatccgagaga

SEQ ID NO: 8 - RNA, 16 nt, synthetic construct
  modified_base 1..3: mod_base = OTHER; note = linked via phosphorothioate
  modified_base 1: mod_base = cm
  modified_base 2: mod_base = gm
  modified_base 3: mod_base = gm
  modified_base 4: mod_base = OTHER; note = 2'-Fluoro-adenosine
  modified_base 5: mod_base = um
  modified_base 6: mod_base = OTHER; note = 2'-Fluoro-cytidine
  modified_base 7: mod_base = um
  modified_base 8: mod_base = OTHER; note = 2'-Fluoro-cytidine
  modified_base 9: mod_base = cm
  modified_base 10: mod_base = OTHER; note = 2'-Fluoro-adenosine
  modified_base 11: mod_base = OTHER; note = 2'-O-methyl-adenosine
  modified_base 12: mod_base = OTHER; note = 2'-O-methyl-adenosine
  modified_base 13: mod_base = um
  modified_base 14..16: mod_base = OTHER; note = linked via phosphorothioate
  modified_base 14: mod_base = OTHER; note = 2'-Fluoro-uridine
  modified_base 15: mod_base = um
  modified_base 16: mod_base = OTHER; note = 2'-O-methyl-adenosine
  source 1..16: mol_type = other RNA
cggatctccaaattta

SEQ ID NO: 9 - RNA, 21 nt, synthetic construct
  modified_base 1..3: mod_base = OTHER; note = linked via phosphorothioate
  modified_base 1: mod_base = um
  modified_base 2: mod_base = OTHER; note = 2'-Fluoro-adenosine
  modified_base 3: mod_base = um
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = um
  modified_base 8: mod_base = OTHER; note = 2'-Fluoro-guanosine
  modified_base 9: mod_base = gm
  modified_base 10: mod_base = OTHER; note = 2'-Fluoro-uridine
  modified_base 11: mod_base = OTHER; note = 2'-O-methyl-adenosine
  modified_base 12: mod_base = OTHER; note = 2'-Fluoro-guanosine
  modified_base 13: mod_base = cm
  modified_base 14..21: mod_base = OTHER; note = linked via phosphorothioate
  modified_base 14: mod_base = OTHER; note = 2'-Fluoro-uridine
  modified_base 15: mod_base = OTHER; note = 2'-O-methyl-adenosine
  modified_base 16: mod_base = OTHER; note = 2'-Fluoro-uridine
  modified_base 17: mod_base = gm
  modified_base 18: mod_base = OTHER; note = 2'-O-methyl-adenosine
  modified_base 19: mod_base = um
  modified_base 20: mod_base = OTHER; note = 2'-Fluoro-guanosine
  modified_base 21: mod_base = OTHER; note = 2'-O-methyl-adenosine
  source 1..21: mol_type = other RNA
tataaatggtagctatgatga

SEQ ID NO: 10 - RNA, 16 nt, synthetic construct
  modified_base 1..3: mod_base = OTHER; note = linked via phosphorothioate
  modified_base 1: mod_base = OTHER; note = 2'-O-methyl-adenosine
  modified_base 2: mod_base = um
  modified_base 3: mod_base = OTHER; note = 2'-O-methyl-adenosine
  modified_base 4: mod_base = OTHER; note = 2'-Fluoro-guanosine
  modified_base 5: mod_base = cm
  modified_base 6: mod_base = OTHER; note = 2'-Fluoro-uridine
  modified_base 7: mod_base = OTHER; note = 2'-O-methyl-adenosine
  modified_base 8: mod_base = OTHER; note = 2'-Fluoro-cytidine
  modified_base 9: mod_base = cm
  modified_base 10: mod_base = OTHER; note = 2'-Fluoro-adenosine
  modified_base 11: mod_base = um
  modified_base 12: mod_base = um
  modified_base 13: mod_base = um
  modified_base 14..16: mod_base = OTHER; note = linked via phosphorothioate
  modified_base 14: mod_base = OTHER; note = 2'-Fluoro-adenosine
  modified_base 15: mod_base = um
  modified_base 16: mod_base = OTHER; note = 2'-O-methyl-adenosine
  source 1..16: mol_type = other RNA
atagctaccatttata

SEQ ID NO: 11 - RNA, 21 nt, synthetic construct
  modified_base 1..3: mod_base = OTHER; note = linked via phosphorothioate
  misc_feature 1: note = 5 -vinylphosphonate moiety attached
  modified_base 1: mod_base = um
  modified_base 2: mod_base = OTHER; note = 2'-Fluoro-adenosine
  modified_base 3: mod_base = OTHER; note = 2'-O-methyl-adenosine
  modified_base 4: mod_base = OTHER; note = 2'-Fluoro-adenosine
  modified_base 5: mod_base = OTHER; note = 2'-Fluoro-uridine
  modified_base 6: mod_base = OTHER; note = 2'-Fluoro-uridine
  modified_base 7: mod_base = um
  modified_base 8: mod_base = OTHER; note = 2'-Fluoro-guanosine
  modified_base 9: mod_base = gm
  modified_base 10: mod_base = OTHER; note = 2'-Fluoro-adenosine
  modified_base 11: mod_base = gm
  modified_base 12: mod_base = OTHER; note = 2'-Fluoro-adenosine
  modified_base 13: mod_base = um
  modified_base 14..21: mod_base = OTHER; note = linked via phosphorothioate
  modified_base 14: mod_base = OTHER; note = 2'-Fluoro-cytidine
  modified_base 15: mod_base = cm
  modified_base 16: mod_base = OTHER; note = 2'-Fluoro-guanosine
  modified_base 17: mod_base = OTHER; note = 2'-O-methyl-adenosine
  modified_base 18: mod_base = gm
  modified_base 19: mod_base = OTHER; note = 2'-O-methyl-adenosine
  modified_base 20: mod_base = OTHER; note = 2'-Fluoro-guanosine
  modified_base 21: mod_base = OTHER; note = 2'-O-methyl-adenosine
  source 1..21: mol_type = other RNA
taaatttggagatccgagaga

SEQ ID NO: 12 - DNA, 18 bp, synthetic construct
  modified_base 1..3: mod_base = OTHER; note = linked via phosphorothioate
  misc_feature 1..16: note = RNA fragment
  modified_base 1: mod_base = cm
  modified_base 2: mod_base = gm
  modified_base 3: mod_base = gm
  modified_base 4: mod_base = OTHER; note = 2'-Fluoro-adenosine
  modified_base 5: mod_base = um
  modified_base 6: mod_base = OTHER; note = 2'-Fluoro-cytidine
  modified_base 7: mod_base = um
  modified_base 8: mod_base = OTHER; note = 2'-Fluoro-cytidine
  modified_base 9: mod_base = cm
  modified_base 10: mod_base = OTHER; note = 2'-Fluoro-adenosine
  modified_base 11: mod_base = OTHER; note = 2'-O-methyl-adenosine
  modified_base 12: mod_base = OTHER; note = 2'-O-methyl-adenosine
  modified_base 13: mod_base = um
  modified_base 14: mod_base = OTHER; note = 2'-Fluoro-uridine
  modified_base 15: mod_base = um
  modified_base 16: mod_base = OTHER; note = 2'-O-methyl-adenosine
  misc_feature 17..18: note = DNA fragment
  misc_feature 18: note = 3'-C7-phosphocholine-docosanoic acid conjugate attached
  source 1..18: mol_type = other DNA
cggatctccaaatttatt

SEQ ID NO: 13 - RNA, 21 nt, synthetic construct
  modified_base 1..3: mod_base = OTHER; note = linked via phosphorothioate
  misc_feature 1: note = 5'-vinylphosphonate moiety attached
  modified_base 1: mod_base = um
  modified_base 2: mod_base = OTHER; note = 2'-Fluoro-adenosine
  modified_base 3: mod_base = um
  modified_base 4: mod_base = OTHER; note = 2'-Fluoro-adenosine
  modified_base 5: mod_base = OTHER; note = 2'-Fluoro-adenosine
  modified_base 6: mod_base = OTHER; note = 2'-Fluoro-adenosine
  modified_base 7: mod_base = um
  modified_base 8: mod_base = OTHER; note = 2'-Fluoro-guanosine
  modified_base 9: mod_base = gm
  modified_base 10: mod_base = OTHER; note = 2'-Fluoro-uridine
  modified_base 11: mod_base = OTHER; note = 2'-O-methyl-adenosine
  modified_base 12: mod_base = OTHER; note = 2'-Fluoro-guanosine
  modified_base 13: mod_base = cm
  modified_base 14..21: mod_base = OTHER; note = linked via phosphorothioate
  modified_base 14: mod_base = OTHER; note = 2'-Fluoro-uridine
  modified_base 15: mod_base = OTHER; note = 2'-O-methyl-adenosine
  modified_base 16: mod_base = OTHER; note = 2'-Fluoro-uridine
  modified_base 17: mod_base = gm
  modified_base 18: mod_base = OTHER; note = 2'-O-methyl-adenosine
  modified_base 19: mod_base = um
  modified_base 20: mod_base = OTHER; note = 2'-Fluoro-guanosine
  modified_base 21: mod_base = OTHER; note = 2'-O-methyl-adenosine
  source 1..21: mol_type = other RNA
tataaatggtagctatgatga

SEQ ID NO: 14 - DNA, 18 bp, synthetic construct
  modified_base 1..3: mod_base = OTHER; note = linked via phosphorothioate
  misc_feature 1..16: note = RNA fragment
  modified_base 1: mod_base = OTHER; note = 2'-O-methyl-adenosine
  modified_base 2: mod_base = um
  modified_base 3: mod_base = OTHER; note = 2'-O-methyl-adenosine
  modified_base 4: mod_base = OTHER; note = 2'-Fluoro-guanosine
  modified_base 5: mod_base = cm
  modified_base 6: mod_base = OTHER; note = 2'-Fluoro-uridine
  modified_base 7: mod_base = OTHER; note = 2'-O-methyl-adenosine
  modified_base 8: mod_base = OTHER; note = 2'-Fluoro-cytidine
  modified_base 9: mod_base = cm
  modified_base 10: mod_base = OTHER; note = 2'-Fluoro-adenosine
  modified_base 11: mod_base = um
  modified_base 12: mod_base = um
  modified_base 13: mod_base = um
  modified_base 14..16: mod_base = OTHER; note = linked via phosphorothioate
  modified_base 14: mod_base = OTHER; note = 2'-Fluoro-adenosine
  modified_base 15: mod_base = um
  modified_base 16: mod_base = OTHER; note = 2'-O-methyl-adenosine
  misc_feature 17..18: note = DNA fragment
  misc_feature 18: note = 3'-C7-phosphocholine-docosanoic acid conjugate attached
  source 1..18: mol_type = other DNA
atagctaccatttatatt

(19) United States
(12) Patent Application Publication
(10) Pub. No.: US 2026/0248839 A1
(43) Pub. Date: Aug. 27, 2026

(54) FORMULATIONS OF NUCLEIC ACID COMPOUNDS AND USES THEREOF

(71) Applicant: Comanche Biopharma Corp., Concord, MA (US)

(72) Inventors: Adel Rafai FAR, Concord, MA (US); Rajeshwar MOTHERAM, Concord, MA (US); Jasmin MONPARA, Secane, PA (US)

(21) Appl. No.: 19/446,532

(22) Filed: Jan. 12, 2026

Related U.S. Application Data
(63) Continuation of application No. PCT/US24/37635, filed on Jul. 11, 2024.
(60) Provisional application No. 63/513,429, filed on Jul. 13, 2023.

(51) Int. Cl. A61K 31/713; A61K 31/724

(52) U.S. Cl. CPC A61K 31/713; A61K 31/724

(57) ABSTRACT

The present disclosure relates to high concentration siRNA compositions suitable for subcutaneous administration. The compositions contain cyclodextrin as a viscosity reducing agent. The present disclosure also relates to methods of treating or preventing a disease using the formulations.

### RELATED APPLICATIONS

This application is a Continuation of International Patent Application No. PCT/US24/37635, filed on Jul. 11, 2024, which claims the benefit of and priority to U.S. Provisional Application No. 63/513,429, filed on Jul. 13, 2023, the entire disclosures of each of which are incorporated herein by reference in their entireties.

### REFERENCE TO AN ELECTRONIC SEQUENCE LISTING

The content of the electronic sequence listing (CMCH_012_001US_SeqList_ST26.xml; Size: 95,210 bytes; and Date of Creation: Jan. 12, 2026) is herein incorporated by reference in its entirety.

### BACKGROUND

Therapeutic administration of siRNA typically requires high doses due to a combination of low cellular uptake and potency. Moreover, the preferred method of siRNA delivery, subcutaneous injection, limits the volume of the administered dose. Hence, highly concentrated formulations are necessary to avoid multiple injections.

However, these formulations may exhibit high viscosity, causing manufacturing challenges, increased tissue back pressure, and injection pain. Highly concentrated oligonucleotide solutions exhibit concentration dependent increase in viscosity which originates from overcrowding of nucleotide strands leading to their self-association. Several reversible interactions including electrostatic, hydrophobic, hydrogen bonding and Vander Waals could contribute to the self-association of nucleotide strands resulting in formation of supra molecular structures. Due to diverse nature of interactions that are specific to nucleotide sequence there is lack of unified theory and mechanistic understanding with regard to development of stable, high concentration nucleic acid formulations with relatively low viscosity.

Thus, there remains a need to develop nucleic acid formulations with high concentrations that exhibit low viscosity, making them suitable for subcutaneous injection.

### SUMMARY

In some aspects, the present disclosure provides a formulation comprising:
(i) a nucleic acid molecule (e.g., a dsRNA molecule); and
(ii) a cyclodextrin agent.

In some aspects, the present disclosure provides a formulation comprising:
(i) two nucleic acid molecules (e.g., a first dsRNA molecule and a second dsRNA molecule); and
(ii) a cyclodextrin agent.

In some aspects, the present disclosure provides a formulation comprising:
(i) a dsRNA molecule, wherein the dsRNA molecule is present at a concentration of at least about 150 mg/mL; and
(ii) a cyclodextrin agent present at a concentration ranging about 5% w/v to about 35% w/v.

In some aspects, the present disclosure provides a formulation comprising:
(ii) a dsRNA molecule, wherein the dsRNA molecule is present at a concentration ranging from about 150 mg/mL to about 250 mg/mL; and
(ii) a cyclodextrin agent present at a concentration ranging about 5% w/v to about 10% w/v, preferably a concentration of about 6.5% w/v.

In some aspects, the present disclosure provides a formulation comprising:
(i) a first dsRNA molecule and a second dsRNA molecule, wherein the first dsRNA molecule and the second dsRNA molecule are present at a total concentration of at least 150 mg/mL; and
(ii) a cyclodextrin agent present at a concentration ranging about 5% w/v to about 35% w/v.

In some aspects, the present disclosure provides a formulation comprising:
(i) a first dsRNA molecule and a second dsRNA molecule, wherein first dsRNA molecule and the second dsRNA molecule are present at a total concentration ranging from about 150 mg/mL to about 250 mg/mL; and
(ii) a cyclodextrin agent present at a concentration ranging about 5% w/v to about 10% w/v, preferably a concentration of about 6.5% w/v.

In some aspects, the present disclosure provides a formulation, comprising:
(i) a dsRNA molecule, wherein the dsRNA molecule is present at a concentration ranging from about 150 mg/mL to about 250 mg/mL; and
(ii) a cyclodextrin agent present in the formulation at a cyclodextrin:dsRNA molar ratio of no more than 5.

In some aspects, the present disclosure provides a method of treating or preventing a disease, comprising administering to a subject in need thereof a formulation disclosed herein.

In some aspects, the present disclosure provides a formulation disclosed herein for use in treating or preventing a disease in a subject in need thereof.

In some aspects, the present disclosure provides a method of inhibiting secreted soluble fms-like tyrosine kinase-1 (sFLT1) in a subject, comprising administering to the subject a formulation disclosed herein.

In some aspects, the present disclosure provides a formulation disclosed herein for use in inhibiting sFLT1 in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF DRAWINGS

FIGS. 1A-1D depict four exemplary formulae of antisense or sense strand of a nucleic acid molecule.

FIGS. 2A-2B depict the viscosity of the formulation containing 1:1 ratio of the first and second dsRNA molecules at a concentration of 150 mg/mL without the addition of HPBCD (FIG. 2A) or with the addition of 6.5% wt of HPBCD (FIG. 2B).

FIG. 3 depicts the viscosity of the formulation containing 1:1 ratio of the first and second dsRNA molecules with various concentrations of the HPBCD.

FIGS. 4A-4C are a set of graphs depicting the UV absorptivity profiles of a first dsRNA molecule (FIG. 4A), a second dsRNA molecule (FIG. 4B), and the combination of the first and second dsRNA molecules (FIG. 4C) when formulated with either water or HPBCD as the medium.

FIG. 5 is a diagram outlining an exemplary process for preparing the formulations.

FIG. 6 is a graph depicting the plasma concentration profile following 50 mg/kg subcutaneous administration of each siRNA (i.e., the first dsRNA and the second dsRNA in 1:1 ratio) in HPBCD and PBS.

### DETAILED DESCRIPTION

The discovery and development of a formulation comprising nucleic acid compounds pose several challenges due to their unique properties. One of the significant challenges is achieving high concentrations of nucleic acid compounds while managing the increase in viscosity that accompanies higher concentrations. As the concentration of nucleic acids increases, their molecular interactions can lead to an elevated viscosity, making it challenging to formulate them into suitable delivery systems.

The present disclosure is based on, inter alia, a discovery that cyclodextrins can remarkably decrease viscosity in highly concentrated oligonucleotide formulations. Traditionally, cyclodextrins have been widely employed as solubility enhancers for hydrophobic molecules. Their ability to form inclusion complexes with hydrophobic moieties allows for improved solubility and bioavailability. However, the discovery that cyclodextrins at concentrations below the commonly utilized levels can also have a substantial impact on reducing viscosity in highly concentrated nucleic acid formulations represents a novel and unexpected finding. This has significant implications for the formulation and development of highly concentrated nucleic acid formulations, particularly for applications where lower viscosity is desired, such as subcutaneous injection. The ability to decrease viscosity while maintaining high concentrations of oligonucleotides can enhance the ease of administration, improve patient comfort, and facilitate efficient delivery of the therapeutic agent.

### Formulations of the Present Disclosure

Provided herein are formulations of nucleic acid molecules, e.g., suitable for dosing or administration by subcutaneous injection.

In some aspects, the present disclosure provides a formulation comprising:
(i) a nucleic acid molecule (e.g., a dsRNA molecule); and
(ii) a cyclodextrin agent.

In some embodiments, the formulation comprises:
(i) two or more nucleic acid molecules (e.g., two or more dsRNA molecules); and
(ii) a cyclodextrin agent.

In some embodiments, the formulation comprises:
(i) two nucleic acid molecules (e.g., a first dsRNA molecule and a second dsRNA molecule); and
(ii) a cyclodextrin agent.

In some embodiments, the formulation further comprises a buffering agent.

In some embodiments, the formulation further comprises a tonicity adjusting agent.

In some aspects, the present disclosure provides a formulation comprising:
(i) two nucleic acid molecules (e.g., a first dsRNA molecule and a second dsRNA molecule);
(ii) a cyclodextrin agent;
(iii) a buffering agent; and
(iv) a tonicity adjusting agent.

In some embodiments, the formulation further comprises one or more excipients.

In some embodiments, the formulation further comprises a solvent.

### Nucleic Acid Agents

In some embodiments, the nucleic acid molecule is suitable for a therapeutic, diagnostic, clinical, or drug delivery use.

In some embodiments, the nucleic acid molecule is a copyDNA (cDNA), a DNA aptamer, a DNAzyme, an RNA aptamer, an external guide sequence, an RNA interference molecule, a morpholino, a messenger RNA (mRNA), or a long non-coding RNA (lincRNA).

In some embodiments, the nucleic acid molecule is an RNA interference molecule.

In some embodiments, the nucleic acid molecule comprises a small interfering RNA (siRNA), a double-stranded RNA (dsRNA), an antisense RNA, a short hairpin RNA, or a micro RNA (miRNA).

In some embodiments, the nucleic acid molecule is a dsRNA molecule.

In some embodiments, the nucleic acid is a high molecular weight nucleic acid with a molecular weight of greater than about 10,000 g/mol. In other embodiments, the nucleic acid is a low molecular weight nucleic acid with a molecular weight of less than about 10,000 g/mol. In some embodiments, the molecular weight of the polymer is determined by methods known in the art, such as mass spectrometry, and the molecular weight figure is determined using methods known in the art as a number average molecular weight, a weight average molecular weight, or a peak average molecular weight. In some embodiments, the nucleic acid has a molecular weight of about 1000-5000 g/mol, or about 5000-10,000 g/mol, or about 5000-15,000 g/mol, or about 10,000-20,000 g/mol.

In some embodiments, the nucleic acid molecules induce gene silencing through RNA interference. Gene expression can also be effectively silenced in a highly specific manner through RNA interference (RNAi).

A small interfering RNA (siRNA) is a double-stranded RNA (dsRNA) that can induce sequence-specific post-transcriptional gene silencing, thereby decreasing or even inhibiting gene expression. In one example, a siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends.

Sequence specific gene silencing can be achieved in mammalian cells using synthetic, short double-stranded RNAs that mimic the siRNAs produced by the enzyme Dicer (Elbashir, et al. (2001) Nature, 411:494 498) (Ui-Tei, et al. (2000) FEBS Lett 479:79-82). siRNA can be chemically or enzymatically-synthesized or can be the result of short double-stranded hairpin-like RNAs (shRNAs) that are processed into siRNAs inside the cell.

In some embodiments, an active agent is a double-stranded RNA (dsRNA) molecule or a pharmaceutically acceptable salt thereof described in WO2022271786A1, the content of which is hereby incorporated by reference in its entirety for any purpose. In some embodiments, the formulation of the present disclosure comprises a dsRNA molecule. In some embodiments, the formulation of the present disclosure comprises two dsRNA molecules (e.g., a first dsRNA molecule and a second dsRNA molecule).

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 50% 2′-O-methyl modifications; (4) the nucleotides at any one or more of positions 2, 4, 5, 6, 8, 10, 12, 14, 16, and 20 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; and (6) a portion of the antisense strand is complementary to a portion of the sense strand.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 50% 2′-O-methyl modifications; (4) the nucleotides at any one or more of positions 2, 4, 5, 6, 8, 10, 12, 14, 16, and 20 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; (6) a portion of the antisense strand is complementary to a portion of the sense strand; (7) the sense strand is at least 15 nucleotides in length; (8) the sense strand comprises at least 65% 2′-O-methyl modifications; (9) the nucleotides at any one of more of positions 4, 6, 8, 10, and 14 from the 5′ end of the sense strand are not 2′-methoxy-ribonucleotides; and (10) the nucleotides at positions 1-2 from the 5′ end of the sense strand are connected to each other via phosphorothioate internucleotide linkages.

In some embodiments, the nucleotides at positions 2, 4, 5, 6, 8, 10, 12, 14, 16, and 20 from the 5′ end of the antisense strand are not 2′-methoxy-ribonucleotides.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand comprises alternating 2′-methoxy-ribonucleotides and 2′-fluoro-ribonucleotides; (3) the nucleotides at positions 2 and 14 from the 5′ end of the antisense strand are not 2′-methoxy-ribonucleotides; (4) the nucleotides at positions 1-2 to 1-7 from the 3′ end of the antisense strand are connected to each other via phosphorothioate internucleotide linkages; and (5) a portion of the antisense strand is complementary to a portion of the sense strand.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand comprises alternating 2′-methoxy-ribonucleotides and 2′-fluoro-ribonucleotides; (3) the nucleotides at positions 2 and 14 from the 5′ end of the antisense strand are not 2′-methoxy-ribonucleotides; (4) the nucleotides at positions 1-2 to 1-7 from the 3′ end of the antisense strand are connected to each other via phosphorothioate internucleotide linkages; (5) a portion of the antisense strand is complementary to a portion of the sense strand; (6) the sense strand comprises alternating 2′-methoxy-ribonucleotides and 2′-fluoro-ribonucleotides; and (7) the nucleotides at positions 1-2 from the 5′ end of the sense strand are connected to each other via phosphorothioate internucleotide linkages.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 50% 2′-O-methyl modifications; (4) the nucleotides at any one or more of positions 2, 4, 5, 6, 8, 10, 12, 14, 16, and 18 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; and (6) a portion of the antisense strand is complementary to a portion of the sense strand.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 50% 2′-O-methyl modifications; (4) the nucleotides at any one or more of positions 2, 4, 5, 6, 8, 10, 12, 14, 16, and 18 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; (6) a portion of the antisense strand is complementary to a portion of the sense strand; (7) the sense strand is at least 15 nucleotides in length; (8) the sense strand comprises at least 80% 2′-O-methyl modifications; (9) the nucleotides at any one of more of positions 7, 9, and 11 from the 5′ end of the sense strand are not 2′-methoxy-ribonucleotides; and (10) the nucleotides at positions 1-2 from the 5′ end of the sense strand are connected to each other via phosphorothioate internucleotide linkages.

In some embodiments, the nucleotides at positions 2, 4, 5, 6, 8, 10, 12, 14, 16, and 18 from the 5′ end of the antisense strand are not 2′-methoxy-ribonucleotides.

In some embodiments, the nucleotides at positions 7, 9, and 11 from the 5′ end of the sense strand are not 2′-methoxy-ribonucleotides.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 70% 2′-O-methyl modifications; (4) the nucleotides at any one or more of positions 2, 4, 5, 6, 8, and 14 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; and (6) a portion of the antisense strand is complementary to a portion of the sense strand.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 70% 2′-O-methyl modifications; (4) the nucleotides at any one or more of positions 2, 4, 5, 6, 8, and 14 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; (6) a portion of the antisense strand is complementary to a portion of the sense strand; (7) the sense strand is at least 15 nucleotides in length; (8) the sense strand comprises 100% 2′-O-methyl modifications; and (9) the nucleotides at positions 1-2 from the 5′ end of the sense strand are connected to each other via phosphorothioate internucleotide linkages.

In some embodiments, the nucleotides at positions 2, 4, 5, 6, 8, and 14 from the 5′ end of the antisense strand are not 2′-methoxy-ribonucleotides.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 75% 2′-O-methyl modifications; (4) the nucleotides at any one or more of positions 2, 4, 5, 6, and 14 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; and (6) a portion of the antisense strand is complementary to a portion of the sense strand.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 75% 2′-O-methyl modifications; (4) the nucleotides at any one or more of positions 2, 4, 5, 6, and 14 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; (6) a portion of the antisense strand is complementary to a portion of the sense strand; (7) the sense strand is at least 15 nucleotides in length; (8) the sense strand comprises 100% 2′-O-methyl modifications; and (9) the nucleotides at positions 1-2 from the 5′ end of the sense strand are connected to each other via phosphorothioate internucleotide linkages.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 85% 2′-O-methyl modifications; (4) the nucleotides at any one or more of positions 2 and 14 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; and (6) a portion of the antisense strand is complementary to a portion of the sense strand.

In some embodiments, the dsRNA molecule comprises an antisense strand and a sense strand, each strand with a 5′ end and a 3′ end, wherein: (1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2); (2) the antisense strand is at least 20 nucleotides in length; (3) the antisense strand comprises at least 85% 2′-O-methyl modifications; (4) the nucleotides at any one or more of positions 2 and 14 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; (6) a portion of the antisense strand is complementary to a portion of the sense strand; (7) the sense strand is at least 15 nucleotides in length; (8) the sense strand comprises 100% 2′-O-methyl modifications; and (9) the nucleotides at positions 1-2 from the 5′ end of the sense strand are connected to each other via phosphorothioate internucleotide linkages.

In some embodiments, the antisense strand is 20 nucleotides in length. In some embodiments, the antisense strand is 21 nucleotides in length. In some embodiments, the antisense strand is 22 nucleotides in length.

In some embodiments, the sense strand is 15 nucleotides in length. In some embodiments, the sense strand is 16 nucleotides in length. In some embodiments, the sense strand is 18 nucleotides in length. In some embodiments, the sense strand is 20 nucleotides in length.

In some embodiments, the dsRNA molecule comprises a double-stranded region of 15 base pairs to 20 base pairs. In some embodiments, the dsRNA molecule comprises a double-stranded region of 15 base pairs. In In some embodiments, the dsRNA molecule comprises a double-stranded region of 16 base pairs. In some embodiments, the dsRNA molecule comprises a double-stranded region of 18 base pairs. In some embodiments, the dsRNA molecule comprises a double-stranded region of 20 base pairs.

In some embodiments, the dsRNA molecule comprises a blunt-end.

In some embodiments, the dsRNA molecule comprises at least one single stranded nucleotide overhang.

In some embodiments, the dsRNA molecule comprises about a 2-nucleotide to 5-nucleotide single stranded nucleotide overhang.

In some embodiments, the dsRNA molecule comprises 4-16 phosphorothioate internucleotide linkages. In some embodiments, the dsRNA molecule comprises 8-13 phosphorothioate internucleotide linkages.

In some embodiments, the sense strand comprises one or more nucleotide mismatches between the antisense strand and the sense strand.

In some embodiments, the antisense strand comprises a 5′ phosphate, a 5′-alkyl phosphonate, a 5′ alkylene phosphonate, or a 5′ alkenyl phosphonate.

In some embodiments, the region of complementarity is complementary to at least 15, 16, 17 or 18 contiguous nucleotides of SEQ ID NO: 1 or SEQ ID NO: 2.

In some embodiments, the region of complementarity contains no more than 3 mismatches with SEQ ID NO: 1 or SEQ ID NO: 2.

In some embodiments, the region of complementarity is fully complementary to SEQ ID NO: 1 or SEQ ID NO: 2.

In some embodiments, the antisense strand comprises (e.g., consists of) the nucleic acid sequence of 5′ UAAAUUUGGAGAUCCGAGAGA 3′ (SEQ ID NO: 3) and the sense strand comprises or consists of the nucleic acid sequence of 5′ CGGAUCUCCAAAUUUA 3′ (SEQ ID NO: 4).

In some embodiments, the antisense strand comprises or consists of the nucleic acid sequence of 5′ UAUAAAUGGUAGCUAUGAUGA 3′ (SEQ ID NO: 5) and the sense strand comprises or consists of the nucleic acid sequence of 5′ AUAGCUACCAUUUAUA 3′ (SEQ ID NO: 6).

In some embodiments, the dsRNA molecule comprises an antisense strand and a sense strand, each strand with a 5′ end and a 3′ end, wherein:

(1) the antisense strand comprises
(SEQ ID NO: 7)
(mU)#(fA)#(mA)(fA)(fU)(fU)(mU)(fG)(mG)(fA)(mG)(fA)
(mU)(fC)#(mC)#(fG)#(mA)#(mG)#(mA)#(fG)#(mA);
and
(2) the sense strand comprises
(SEQ ID NO: 8)
(mC)#(mG)#(mG)(fA)(mU)(fC)(mU)(fC)(mC)(fA)(mA)(mA)
(mU)(fU)#(mU)#(mA),

wherein “m” corresponds to a 2′-O-methyl modification, “f” corresponds to a 2′-fluoro modification, “#” corresponds to a phosphorothioate internucleotide linkage.

In some aspects, the dsRNA molecule comprising an antisense strand and a sense strand, each strand with a 5′ end and a 3′ end, wherein:

(1) the antisense strand comprises
(SEQ ID NO: 9)
(mU)#(fA)#(mU)(fA)(fA)(fA)(mU)(fG)(mG)(fU)(mA)(fG)
(mC)(fU)#(mA)#(fU)#(mG)#(mA)#(mU)#(fG)#(mA);
and
(2) the sense strand comprises
(SEQ ID NO: 10)
(mA)#(mU)#(mA)(fG)(mC)(fU)(mA)(fC)(mC)(fA)(mU)(mU)
(mU)(fA)#(mU)#(mA),

wherein “m” corresponds to a 2′-O-methyl modification, “f” corresponds to a 2′-fluoro modification, “#” corresponds to a phosphorothioate internucleotide linkage.

In some embodiments, the antisense strand comprises a 5′ vinyl phosphonate.

In some embodiments, a functional moiety is linked to the 3′ end of the sense strand.

In some embodiments, the functional moiety comprises a hydrophobic moiety.

In some embodiments, the hydrophobic moiety is selected from the group consisting of fatty acids, steroids, secosteroids, lipids, gangliosides, nucleoside analogs, endocannabinoids, vitamins, and a mixture thereof.

In some embodiments, the steroid selected from the group consisting of cholesterol and Lithocholic acid (LCA).

In some embodiments, the fatty acid selected from the group consisting of Eicosapentaenoic acid (EPA), Docosahexaenoic acid (DHA) and Docosanoic acid (DCA). In some embodiments, the fatty acid is EPA. In some embodiments, the fatty acid is DHA. In some embodiments, the fatty acid is DCA. In some embodiments, the fatty acid is PC-DCA.

In some embodiments, the vitamin is selected from the group consisting of choline, vitamin A, vitamin E, and derivatives or metabolites thereof.

In some embodiments, the functional moiety is linked to the sense strand by a linker.

In some embodiments, the dsRNA molecule comprises a docosanoic acid (DCA) conjugate linked to the 3′ end of the sense strand.

In some embodiments, the DCA is linked to the sense strand by a linker

In some embodiments, the linker is a cleavable linker.

In some embodiments, the cleavable linker comprises a phosphodiester linkage, a disulfide linkage, an acid-labile linkage, or a photocleavable linkage.

In some embodiments, the cleavable linker comprises a dTdT dinucleotide with phosphodiester internucleotide linkages.

In some embodiments, the acid-labile linkage comprises a β-thiopropionate linkage or a carboxydimethylmaleic anhydride (CDM) linkage.

In some embodiments, the linker comprises a divalent or trivalent linker.

In some embodiments, the divalent or trivalent linker is

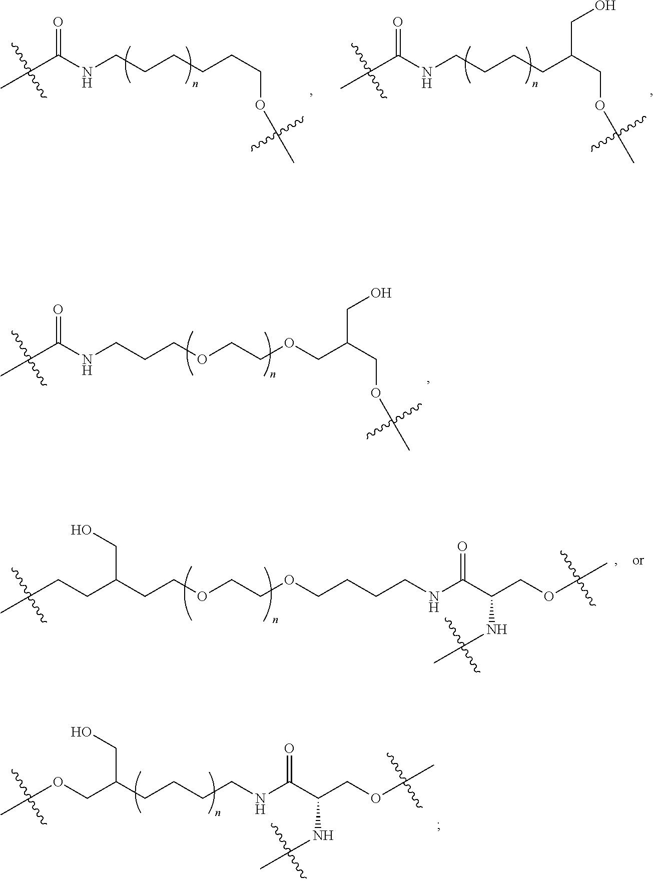

wherein n is 1, 2, 3, 4, or 5.

In some embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In some embodiments, when the linker is a trivalent linker, the linker further links a phosphodiester or phosphodiester derivative.

In some embodiments, the linker further links a phosphodiester or phosphodiester derivative.

In some embodiments, the phosphodiester or phosphodiester derivative is

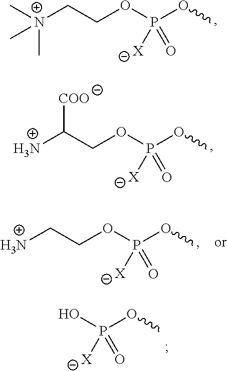

wherein X is O, S or BH₃.

In some embodiments, the nucleotides at positions 1 and 2 from the 3′ end of sense strand, and the nucleotides at positions 1 and 2 from the 5′ end of antisense strand, are connected to adjacent ribonucleotides via phosphorothioate linkages.

In some embodiments, the dsRNA molecule comprises an antisense strand and a sense strand, each strand with a 5′ end and a 3′ end, wherein:

(1) the antisense strand comprises
(SEQ ID NO: 11)
V(mU)#(fA)#(mA)(fA)(fU)(fU)(mU)(fG)(mG)(fA)(mG)
(fA)(mU)(fC)#(mC)#(fG)#(mA)#(mG)#(mA)#(fG)#(mA);
and
(2) the sense strand comprises
(SEQ ID NO: 12)
(mC)#(mG)#(mG)(fA)(mU)(fC)(mU)(fC)(mC)(fA)(mA)
(mA)(mU)(fU)#(mU)#(mA)(T)(T)-PCDCA,

wherein “m” corresponds to a 2′-O-methyl modification, “f” corresponds to a 2′-fluoro modification, “T” corresponds to a thymidine DNA nucleotide, “#” corresponds to a phosphorothioate internucleotide linkage, “V” corresponds to a 5′-vinylphosphonate, and “PCDCA” corresponds to a 3′-C7-phosphocholine-docosanoic acid conjugate through a phosphate linker.

In some embodiments, the dsRNA molecule comprises an antisense strand and a sense strand, each strand with a 5′ end and a 3′ end, wherein:

(1) the antisense strand comprises
(SEQ ID NO: 13)
V(mU)#(fA)#(mU)(fA)(fA)(fA)(mU)(fG)(mG)(fU)(mA)
(fG)(mC)(fU)#(mA)#(fU)#(mG)#(mA)#(mU)#(fG)#(mA);
and
(2) the sense strand comprises
(SEQ ID NO: 14)
(mA)#(mU)#(mA)(fG)(mC)(fU)(mA)(fC)(mC)(fA)(mU)
(mU)(mU)(fA)#(mU)#(mA)(T)(T)-PCDCA,

wherein “m” corresponds to a 2′-O-methyl modification, “f” corresponds to a 2′-fluoro modification, “T” corresponds to a thymidine DNA nucleotide, “#” corresponds to a phosphorothioate internucleotide linkage, “V” corresponds to a 5′-vinylphosphonate, and “PCDCA” corresponds to a 3′-C7-phosphocholine-docosanoic acid conjugate through a phosphate linker.

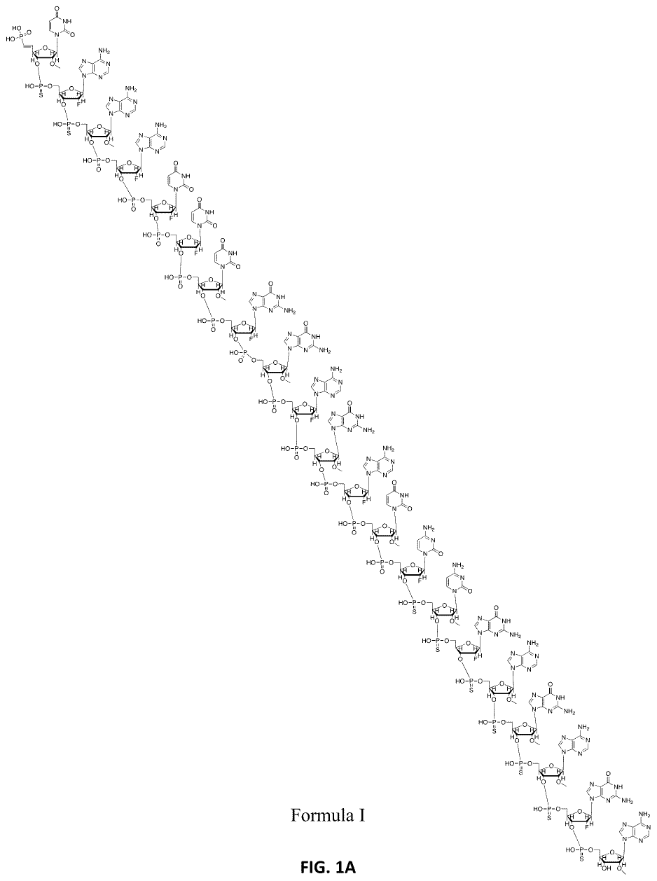

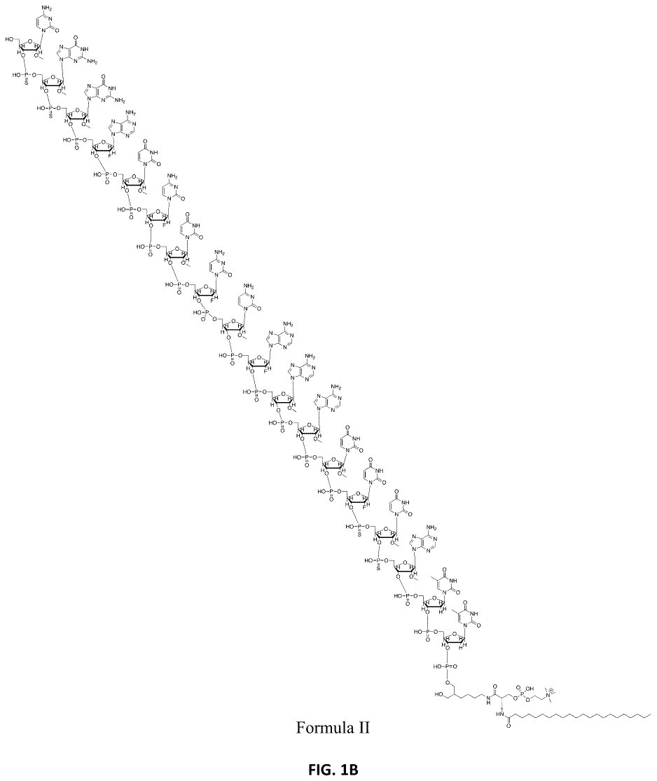

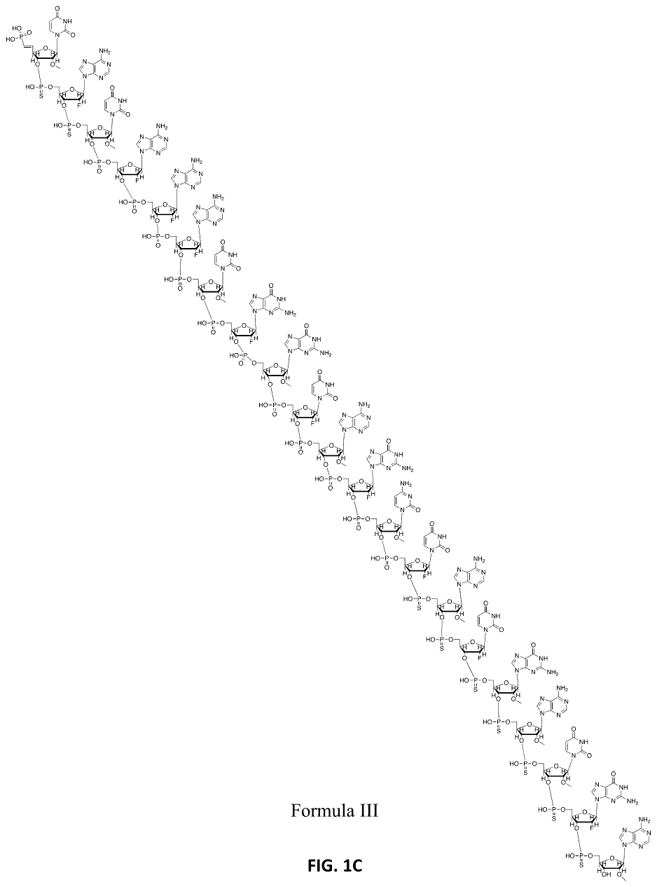

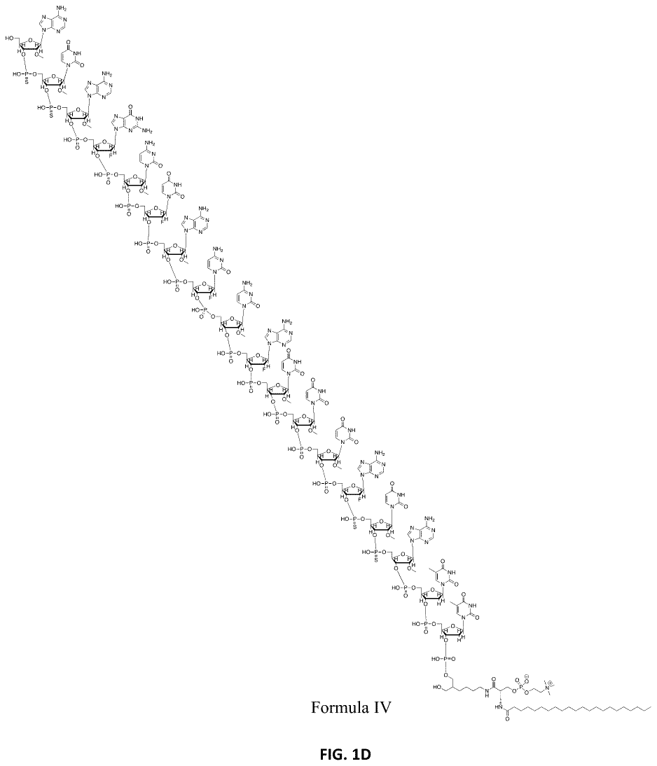

In some embodiments, the dsRNA molecule comprises an antisense strand and a sense strand, each strand with a 5′ end and a 3′ end, wherein:
(1) the antisense strand comprises Formula I (FIG. 1A), or a salt thereof; and
(2) the sense strand comprises Formula II (FIG. 1B), or a salt thereof.

In some embodiments, the dsRNA molecule comprises an antisense strand and a sense strand, each strand with a 5′ end and a 3′ end, wherein:
(1) the antisense strand comprises Formula III (FIG. 1C), or a salt thereof; and
(2) the sense strand comprises Formula IV (FIG. 1D), or a salt thereof.

In some embodiments, the dsRNA molecule is a neutral molecule, an ionic form thereof, or a salt thereof.

In some embodiments, the dsRNA molecule is a pharmaceutically acceptable salt.

In some embodiments, the dsRNA molecule is a sodium salt or a potassium salt.

In some embodiments, the dsRNA molecule is a sodium salt.

In some embodiments, the dsRNA molecule is a potassium salt.

First and Second dsRNA Molecules

In some embodiments, the formulation comprises two or more dsRNA molecules.

In some embodiments, the formulation comprises:
a first dsRNA molecule comprising a first sense strand and a first antisense strand, wherein the first antisense strand comprises a region of complementarity which is substantially complementary to SEQ ID NO: 1; and
a second dsRNA molecule comprising a second sense strand and a second antisense strand, wherein the second antisense strand comprises a region of complementarity which is substantially complementary to SEQ ID NO: 2.

In some embodiments, the formulation comprises:
a first dsRNA molecule comprising a first sense strand and a first antisense strand, each strand with a 5′ end and a 3′ end, wherein the first antisense strand comprises a region of complementarity which is substantially complementary to SEQ ID NO: 1; and
a second dsRNA molecule comprising a second sense strand and a second antisense strand, each strand with a 5′ end and a 3′ end, wherein the second antisense strand comprises a region of complementarity which is substantially complementary to SEQ ID NO: 2;
wherein for each of the first dsRNA molecule and second dsRNA molecule: (1) the antisense strand is at least 20 nucleotides in length; (3) the antisense strand comprises at least 50% 2′-O-methyl modifications; (4) the nucleotides at any one or more of positions 2, 4, 5, 6, 8, 10, 12, 14, 16, and 20 from the 5′ end of the antisense strand are not 2′-methoxy-ribonucleotides; (5) the nucleotides at positions 1-2 to 1-8 from the 3′ end of the antisense strand are connected to each other via phosphorothioate internucleotide linkages; (6) a portion of the antisense strand is complementary to a portion of the sense strand; (7) the sense strand is at least 15 nucleotides in length; (8) the sense strand comprises at least 65% 2′-O-methyl modifications; (9) the nucleotides at any one of more of positions 4, 6, 8, 10, and 14 from the 5′ end of the sense strand are not 2′-methoxy-ribonucleotides; and (10) the nucleotides at positions 1-2 from the 5′ end of the sense strand are connected to each other via phosphorothioate internucleotide linkages.

In some embodiments, the formulation comprises:
(a) a first dsRNA molecule comprises a first antisense strand and a first sense strand, each strand with a 5′ end and a 3′ end, wherein:

(a-1) the first antisense strand comprises
(SEQ ID NO: 7)
(mU)#(fA)#(mA)(fA)(fU)(fU)(mU)(fG)(mG)(fA)(mG)(fA)
(mU)(fC)#(mC)#(fG)#(mA)#(mG)#(mA)#(fG)#(mA);
and
(a-2) the first sense strand comprises
(SEQ ID NO: 8)
(mC)#(mG)#(mG)(fA)(mU)(fC)(mU)(fC)(mC)(fA)(mA)(mA)
(mU)(fU)#(mU)#(mA),

(b) a second dsRNA molecule comprising a second antisense strand and a second sense strand, each strand with a 5′ end and a 3′ end, wherein:

(b-1) the second antisense strand comprises
(SEQ ID NO: 9)
(mU)#(fA)#(mU)(fA)(fA)(fA)(mU)(fG)(mG)(fU)(mA)(fG)
(mC)(fU)#(mA)#(fU)#(mG)#(mA)#(mU)#(fG)#(mA);
and
(b-2) the second sense strand comprises
(SEQ ID NO: 10)
(mA)#(mU)#(mA)(fG)(mC)(fU)(mA)(fC)(mC)(fA)(mU)(mU)
(mU)(fA)#(mU)#(mA),

wherein “m” corresponds to a 2′-O-methyl modification, “f” corresponds to a 2′-fluoro modification, “#” corresponds to a phosphorothioate internucleotide linkage.

In some embodiments, the formulation comprises:
(a) a first dsRNA molecule comprises a first antisense strand and a first sense strand, each strand with a 5′ end and a 3′ end, wherein:

(a-1) the first antisense strand comprises
(SEQ ID NO: 11)
V(mU)#(fA)#(mA)(fA)(fU)(fU)(mU)(fG)(mG)(fA)(mG)
(fA)(mU)(fC)#(mC)#(fG)#(mA)#(mG)#(mA)#(fG)#(mA);
and
(a-2) the first sense strand comprises
(SEQ ID NO: 12)
(mC)#(mG)#(mG)(fA)(mU)(fC)(mU)(fC)(mC)(fA)(mA)(mA)
(mU)(fU)#(mU)#(mA)(T)(T)-PCDCA,

(b) a second dsRNA molecule comprises a second antisense strand and a second sense strand, each strand with a 5′ end and a 3′ end, wherein:

(b-1) the second antisense strand comprises
(SEQ ID NO: 13)
V(mU)#(fA)#(mU)(fA)(fA)(fA)(mU)(fG)(mG)(fU)(mA)
(fG)(mC)(fU)#(mA)#(fU)#(mG)#(mA)#(mU)#(fG)#(mA);
and
(b-2) the second sense strand comprises
(SEQ ID NO: 14)
(mA)#(mU)#(mA)(fG)(mC)(fU)(mA)(fC)(mC)(fA)(mU)(mU)
(mU)(fA)#(mU)#(mA)(T)(T)-PCDCA,

wherein “m” corresponds to a 2′-O-methyl modification, “f” corresponds to a 2′-fluoro modification, “T” corresponds to a thymidine DNA nucleotide, “#” corresponds to a phosphorothioate internucleotide linkage, “V” corresponds to a 5′-vinylphosphonate, and “PCDCA” corresponds to a 3′-C7-phosphocholine-docosanoic acid conjugate through a phosphate linker.

In some embodiments, the formulation comprises:
(a) a first dsRNA, said first dsRNA comprising a first antisense strand and a first sense strand, each strand with a 5′ end and a 3′ end, wherein:
   (a-1) the first antisense strand comprises Formula I (FIG. 1A) or a salt thereof; and
   (a-2) the sense strand comprises Formula II (FIG. 1B) or a salt thereof; and
(b) a second dsRNA, said second dsRNA comprising a second antisense strand and a second sense strand, each strand with a 5′ end and a 3′ end, wherein:
   (b-1) the second antisense strand comprises Formula III (FIG. 1C) or a salt thereof; and
   (b-2) the second sense strand comprises Formula IV (FIG. 1D) or a salt thereof.

Table 1 provides the molecular formulae, weights and sequence of the exemplary first
and second dsRNA molecules.

| | | | Molecular | | Molecular |
|---|---|---|---|---|---|
| | | Molecular | weight of | Molecular | weight of |
| | | Formula of | the sodium | Formula | the free |
| | | the sodium | salt | of the free | acid |
| | Sequence | salt | (g/mol) | acid | (g/mol) |
| First dsRNA | | C₄₃₄H₅₂₇F₁₅N₁₅₀ | 14,705.1 | C₄₃₄H₅₆₇F₁₅ | 13,825.8 |
| | | Na₄₀O₂₅₀P₄₀S₁₃ | | N₁₅₀O₂₅₀P₄₀ | |
| | | | | S₁₃ | |
| Antisense stand of | V(mU)#(fA)#(mA) | C₂₁₅H₂₃₉F₁₀N₈₆ | 7,662.5 | C₂₁₅H₂₆₁F₁₀ | 7,178.9 |
| the first dsRNA | (fA)(fU)(fU)(mU) | Na₂₂O₁₂₅P₂₁S9 | | N₈₆O₁₂₅P₂₁ | |
| | (fG)(mG)(fA)(mG) | | | S₉ | |
| | (fA)(mU)(fC)#(mC)# | | | | |
| | (fG)#(mA)#(mG)# | | | | |
| | (mA)#(fG)#(mA) | | | | |
| Sense strand of the | (mC)#(mG)#(mG) | C₂₁₉H₂₈₈F₅N₆₄ | 7,042.6 | C₂₁₉H₃₀₆F₅ | 6,646.9 |
| first dsRNA | (fA)(mU)(fC)(mU) | Na₁₈O₁₂₅P₁₉S₄ | | N₆₄O₁₂₅P₁₉ | |
| | (fC)(mC)(fA)(mA) | | | S₄ | |
| | (mA)(mU)(fU)# | | | | |
| | (mU)#(mA)(T)(T)- | | | | |
| | PCDCA | | | | |
| Second dsRNA | | C₄₃₃H₅₂₄F₁₅N₁₄₅ | 14,652.0 | C₄₃₃H₅₆₄F₁₅ | 13,772.7 |
| | | Na₄₀O₂₅₂P₄₀S₁₃ | | N₁₄₅O₂₅₂P₄₀ | |
| | | | | S₁₃ | |
| Antisense stand of | V(mU)#(fA)#(mU) | C₂₁₄H₂₃₇F₁₀N₈₂ | 7,624.5 | C₂₁₄H₂₅₉F₁₀ | 7,140.9 |
| the second dsRNA | (fA)(fA)(fA)(mU) | Na₂₂O₁₂₇P₂₁S₉ | | N₈₂O₁₂₇P₂₁ | |
| | (fG)(mG)(fU)(mA) | | | S₉ | |
| | (fG)(mC)(fU)#(mA) | | | | |
| | #(fU)#(mG)#(mA) | | | | |
| | #(mU)#(fG)#(mA) | | | | |
| Sense strand of the | (mA)#(mU)#(mA) | C₂₁₉H₂₈₇F₅N₆₃ | 7,027.6 | C₂₁₉H₃₀₅F₅ | 6,631.9 |
| second dsRNA | (fG)(mC)(fU)(mA) | Na₁₈O₁₂₅P₁₉S₄ | | N₆₃O₁₂₅P₁₉ | |
| | (fC)(mC)(fA)(mU) | | | S₄ | |
| | (mU)(mU)(fA)# | | | | |
| | (mU)#(mA)(T)(T)- | | | | |
| | PCDCA | | | | |

m = 2′-O-methyl
f = 2′-fluoro
T = Thymidine
# = Phosphorothioate
V = 5′-Vinylphosphonate
PCDCA = 3′-C7-Phosphocholine-docosanoic acid conjugate through phosphate linker

Concentrations of Nucleic Acid Molecules (dsRNA Molecules)

In some embodiments, the nucleic acid molecules (e.g., dsRNA molecules) are present in the formulation at a total concentration of about 100 mg/mL or greater, about 110 mg/mL or greater, about 120 mg/mL or greater, about 130 mg/mL or greater, about 140 mg/mL or greater, about 150 mg/mL or greater, about 160 mg/mL or greater, about 170 mg/mL or greater, about 180 mg/mL or greater, about 190 mg/mL or greater, about 200 mg/mL or greater, about 250 mg/mL or greater, about 300 mg/mL or greater, about 350 mg/mL or greater, about 400 mg/mL or greater, about 450 mg/mL or greater, or about 500 mg/mL or greater.

In some embodiments, the nucleic acid molecules (e.g., dsRNA molecules) are present in the formulation at a total concentration of about 100 mg/mL, about 105 mg/mL, about 110 mg/L, about 115 mg/mL, about 120 mg/L, about 125 mg/mL, about 130 mg/mL, about 135 mg/mL, about 140 mg/L, about 145 mg/L, about 150 mg/L, about 155 mg/L, about 160 mg/mL, about 165 mg/mL, about 170 mg/L, about 175 mg/L, about 180 mg/L, about 185 mg/L, about 190 mg/mL, about 195 mg/mL, about 200 mg/L, about 205 mg/L, about 210 mg/L, about 215 mg/L, about 220 mg/L, about 225 mg/L, about 230 mg/L, about 235 mg/L, about 240 mg/L, about 245 mg/L, or about 250 mg/L.

In some embodiments, the first dsRNA molecule and the second dsRNA molecule are present in the formulation at a total concentration of about 100 mg/mL, about 105 mg/mL, about 110 mg/L, about 115 mg/mL, about 120 mg/L, about 125 mg/mL, about 130 mg/mL, about 135 mg/mL, about 140 mg/L, about 145 mg/L, about 150 mg/L, about 155 mg/L, about 160 mg/mL, about 165 mg/mL, about 170 mg/L, about 175 mg/L, about 180 mg/L, about 185 mg/L, about 190 mg/mL, about 195 mg/mL, about 200 mg/L, about 205 mg/L, about 210 mg/L, about 215 mg/L, about 220 mg/L, about 225 mg/L, about 230 mg/L, about 235 mg/L, about 240 mg/L, about 245 mg/L, or about 250 mg/L.

In some embodiments, the first dsRNA molecule is present in the formulation at a concentration of about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, or about 125 mg/mL.

In some embodiments, the second dsRNA molecule is present in the formulation at a concentration of about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, or about 125 mg/mL.

In some embodiments, the first dsRNA molecule is present in the formulation at a concentration of about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, or about 125 mg/mL; and the second dsRNA molecule is present in the formulation at a concentration of about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, or about 125 mg/mL.

In some embodiments, the first dsRNA molecule is present in the formulation at a concentration of about 87.5±30 mg/mL, about 87.5±25 mg/mL, about 87.5±20 mg/mL, about 87.5±15 mg/mL, about 87.5±10 mg/mL, about 87.5±5 mg/mL, about 87.5±4 mg/mL, about 87.5±3 mg/mL, about 87.5±2 mg/mL, or about 87.5±1 mg/mL (e.g., about 87.5 mg/mL).

In some embodiments, the second dsRNA molecule is present in the formulation at a concentration of about 87.5±30 mg/mL, about 87.5±25 mg/mL, about 87.5±20 mg/mL, about 87.5±15 mg/mL, about 87.5±10 mg/mL, about 87.5±5 mg/mL, about 87.5±4 mg/mL, about 87.5±3 mg/mL, about 87.5±2 mg/mL, or about 87.5±1 mg/mL (e.g., about 87.5 mg/mL).

In some embodiments, the first dsRNA molecule is present in the formulation at a concentration of about 87.5±30 mg/mL, about 87.5±25 mg/mL, about 87.5±20 mg/mL, about 87.5±15 mg/mL, about 87.5±10 mg/mL, about 87.5±5 mg/mL, about 87.5±4 mg/mL, about 87.5±3 mg/mL, about 87.5±2 mg/mL, or about 87.5±1 mg/mL (e.g., about 87.5 mg/mL); and the second dsRNA molecule is present in the formulation at a concentration of about 87.5±30 mg/mL, about 87.5±25 mg/mL, about 87.5±20 mg/mL, about 87.5±15 mg/mL, about 87.5±10 mg/mL, about 87.5±5 mg/mL, about 87.5±4 mg/mL, about 87.5±3 mg/mL, about 87.5±2 mg/mL, or about 87.5±1 mg/mL (e.g., about 87.5 mg/mL).

### Cyclodextrin Agents

Without wishing to be bound by theory, it is understood that cyclodextrins could significantly decrease viscosity (e.g., by up to nine times) of the formulation comprising the nucleic acid molecules. Such viscosity reduction could be observed with cyclodextrins at a significantly lower concentrations as compared to the concentration of cyclodextrins when typically used to augment solubility of molecules.

In some embodiments, the cyclodextrin agent is a substituted or unsubstituted cyclodextrin.

In some embodiments, the cyclodextrin agent is a substituted or unsubstituted α-cyclodextrin, a substituted or unsubstituted β-cyclodextrin, or a substituted or unsubstituted γ-cyclodextrin.

Cyclodextrins are non-reducing cyclic glucose oligosaccharides produced from starch. There are three common cyclodextrins with 6, 7 or 8 glucose units (α-, β-, and γ-cyclodextrin respectively) linked by α-1,4 glycosidic bonds. Cyclodextrins can act as molecular containers by entrapping guest molecules in their internal cavity thereby forming inclusion complexes. The α-cyclodextrins have a smaller cavity whereas the β-, and γ-cyclodextrins have larger cavities.

Suitable cyclodextrins include alpha, beta, and gamma cyclodextrins, although beta and gamma cyclodextrins are preferred given their larger internal cavities. Chemical modifications have been made to the cyclodextrins, particularly the β-cyclodextrins to improve the solubility of the parent cyclodextrin. Hydroxyethyl β-cyclodextrin, hydroxypropyl β-cyclodextrin (e.g., 2-Hydroxypropyl-β-cyclodextrin), methylated β-cyclodextrin, glucosyl β-cyclodextrin, and sulfobutyl ether β-cyclodextrin are examples of cyclodextrins that have been chemically modified to improve their solubility. In some embodiments, a β-cyclodextrin including chemically modified β-cyclodextrins will be used. In some embodiments, the chemically modified β-cyclodextrin will be hydroxypropyl β-cyclodextrin or sulfobutylether β-cyclodextrin. In some embodiments, the cyclodextrin will be a gamma cyclodextrin including chemically modified gamma cyclodextrins. In some embodiments, cyclodextrin will be a hydroxypropyl cyclodextrin (e.g., HP4.3-β-cyclodextrin, HP5.5-β-cyclodextrin, HP7.6-β-cyclodextrin, and HP4.5-γ-cyclodextrin). In some embodiments, the cyclodextrin will be a sulfobuytlether β-cyclodextrins (e.g., SBE6.6-β-cyclodextrin, SBE6.7-β-cyclodextrin, SBE6.8-β-cyclodextrin, SBE4.1-β-cyclodextrin, and SBE4.6Et3.5-β-cyclodextrin). In some embodiments, the cyclodextrin will be a sulfobuytlether γ-cyclodextrins (e.g., SBE4.3-γ-cyclodextrin, SBE4.6-γ-cyclodextrin, SBE5.2-γ-cyclodextrin, and SBE5.6Et6.3-γ-cyclodextrin). As used herein “a chemically modified beta cyclodextrin” is a beta cyclodextrin that has been chemically modified to at least have improved solubility as compared to its parent cyclodextrin (i.e., the unmodified cyclodextrin).

In some embodiments, the cyclodextrin agent is a substituted or unsubstituted 3-cyclodextrin.

In some embodiments, the cyclodextrin agent is an unsubstituted β-cyclodextrin.

In some embodiments, the cyclodextrin agent is a substituted β-cyclodextrin.

The hydroxypropyl (HP) substitution for β-cyclodextrin (BCD) refers to the modification where hydroxypropyl groups are attached to the BCD molecule. The level of HP substitution can vary and is typically categorized as low, medium, or high. This level essentially refers to the average number of hydroxypropyl groups added per cyclodextrin molecule. For medium molar hydroxypropyl substitution, the nominal substitution value for HPBCD is about 0.61; for high molar hydroxypropyl substation, the nominal substitution value for HPBCD is about 0.9.

In some embodiments, the cyclodextrin agent is hydroxypropyl β-cyclodextrin (HPBCD).

In some embodiments, the w/v of HPBCD is from 5% to 10%.

In some embodiments, the cyclodextrin agent (e.g., HPBCD) is present in the formulations at a concentration of about 5% w/v or greater.

In some embodiments, HPBCD is present in the formulation at a concentration ranging from about 5% w/v to about 10% w/v.

In some embodiments, HPBCD is present in the formulation at a concentration of about 5.0% w/v, about 5.5% w/v, about 6.0% w/v, about 6.5% w/v, about 7.0% w/v, about 7.5% w/v, about 8% w/v, about 8.5% w/v, about 9% w/v, about 9.5% w/v, or about 10% w/v.

In some embodiments, HPBCD is present in the formulation at a concentration of about 6.5±3.0% w/v, about 6.5±2.5% w/v, about 6.5±2.0% w/v, about 6.5±1.0% w/v, about 6.5±0.9% w/v, about 6.5±0.8% w/v, about 6.5±0.7% w/v, about 6.5±0.6% w/v, about 6.5±0.5% w/v, about 6.5±0.4% w/v, about 6.5±0.3% w/v, about 6.5±0.2% w/v, or about 6.5±0.1% w/v.

In some embodiments, HPBCD is present in the formulation at a concentration of about 6.5% w/v.

In some embodiments, HPBCD is present in the formulation at a concentration no more than 10% w/v.

In some embodiments, HPBCD is present in the formulation at a concentration of about 65±30 mg/mL, about 65±25 mg/mL, about 65±20 mg/mL, about 65±15 mg/mL, about 65±10 mg/mL, about 65±5 mg/mL, about 65±4 mg/mL, about 65±3 mg/mL, about 65±2 mg/mL, or about 65±1 mg/mL (e.g., about 65 mg/mL).

In some embodiments, the HPBCD:dsRNA molar ratio in the formulation is no more than 5. In some embodiments, the HPBCD:dsRNA molar ratio in the formulation is between 2.5 and 5. In some embodiments, the HPBCD:dsRNA molar ratio in the formulation is about 2.5, about 3.0, about 3.5, about 4.0, about 4.5, or about 5.0. In some embodiments, the HPBCD:dsRNA molar ratio in the formulation is between 3 and 15, between 5 and 13, between 6 and 11, or between 6.5 and 10 In some embodiments, the HPBCD:dsRNA molar ratio in the formulation is about 3.0, about 4.0, about 5.0, about 6.0, about 7.0, about 8.0, about 9.0, about 10.0, about 11.0, about 12.0, about 13.0, about 14.0, or about 15.0.

### Buffering Agents

Without wishing to be bound by theory, the buffering agent may maintain a desired pH range of the formulation.

Pharmaceutically acceptable buffering agents are well known in the art, and include without limitation, phosphate buffers, histidine, sodium citrate, HEPES, Tris, Bicine, glycine, N-glycylglycine, sodium acetate, sodium carbonate, glycylglycine, lysine, arginine, sodium phosphate, and mixtures thereof. In some embodiments, the buffer is selected from histidine, phosphate buffer, HEPES, and sodium citrate. In some embodiments, the buffering agent is selected from sodium phosphate dibasic, potassium phosphate monobasic, or any combination thereof.

In some embodiments, the buffering agent is present in the formulation at a concentration of about 0.10% w/v, about 0.09% w/v, about 0.08% w/v, about 0.07% w/v, about 0.06% w/v, about 0.05% w/v, about 0.04% w/v, about 0.03% w/v, about 0.02% w/v, or about 0.01% w/v.

Exemplary concentrations of buffers for formulations of the present disclosure are from about 5 mM to about 100 mM, about 50 mM, about 10 mM to about 40 mM, or about 20 mM. With some embodiments, Tris is included at about 5 mM to about 50 mM, about 10 mM to about 40 mM, or about 20 mM.

In some embodiments, the formulation has a pH value ranging from about 6.5 to about 9.0.

In some embodiments, the formulation has a pH value of about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0.

In some embodiments, the formulation has a pH value ranging from about 6.0 to about 8.0.

In some embodiments, the formulation has a pH value ranging from about 6.5 to about 7.5.

In some embodiments, the formulation has a pH value of about 7.0±2.0, about 7.0±1.5, about 7.0±1.0, about 7.0±0.9, about 7.0±0.8, about 7.0±0.7, about 7.0±0.6, about 7.0±0.5, about 7.0±0.4, about 7.0±0.3, about 7.0±0.2, or about 7.0±0.1 (e.g., about 7.0).

### Tonicity Adjusting Agents

In some embodiments, the formulation comprises a tonicity adjusting agent, also known as an osmotic agent or osmolarity modifier to adjust and maintain the osmotic pressure or tonicity of the formulation to match that of the targeted biological environment or target tissue.

In some embodiments, the tonicity adjusting agent can be selected from a wide variety of tonicity adjusting agents including sodium chloride, potassium chloride, dextrose, mannitol, glycerin, and sorbitol. In some embodiments, the tonicity adjusting agent is selected from sodium chloride, potassium chloride, and any combination thereof.

In some embodiments, the tonicity adjusting agent is present in the formulation at a concentration of about 0.50% w/v, about 0.45% w/v, about 0.40% w/v, about 0.35% w/v, about 0.30% w/v, about 0.25% w/v, about 0.20% w/v, about 0.15% w/v, or about 0.10% w/v.

Exemplary concentrations of tonicity adjusting agents for formulations of the present disclosure are from about 50 mM, about 100 mM, about 150 mM, about 200 mM, about 250 mM, or about 300 mM.

### Solvents and Other Suitable Excipients

In some embodiments, the solvent comprises water.

In some embodiments, the solvent is water.

In some embodiments, the excipient comprises a preservative, a humectant, or a chelating agent.

In some embodiments, the preservative comprises sorbic acid, a paraben (e.g., methyl paraben or propyl paraben), sodium methyl paraben, sodium propyl paraben, sorbitol solution, thimersal, quaternary ammonium salts (NH₄ salts), benzalkonium chloride, potassium permanganate, cealkonium chloride, cetyl pyridinium chloride, cetrimide, quaternium-15, sodium benzoate, imidurea, diazolidinyl urea, chlorhexidine gluconoate, urea, DMDM hydantoin, isochlorthiozolines, benzoic acid, benzyl alcohol, phenoxyethanol, or any combination thereof (e.g., at a concentration ranging from about 0.1% w/w to about 10% w/w).

In some embodiments, the preservative comprises sorbic acid, methyl paraben, and propyl paraben.

In some embodiments, the humectant comprises glycerin, sorbitol, propylene glycol, butylene glycol, hexylene glycol, (polyethylene glycol)ₙ where n=200, 300, 400, 550, 600, or 1000, water, or a combination thereof (e.g., at a concentration ranging from about 0.1% w/w to about 50% w/w).

In some embodiments, the chelating agent comprises ethylenediaminetetraacetic acid (EDTA), disodium edetate, calcium EDTA, or a combination thereof (e.g., at a concentration ranging from about 0.1% w/w to about 10% w/w).

### Properties of the Formulations

In some embodiments, upon administration (e.g., subcutaneous administration) to a subject, the formulation exhibits improved pharmacokinetic profile, as compared to a reference formulation (e.g., without HPBCD). Without being bound by theory, the improved pharmacokinetic profile of the formulation may result in reduced local interaction of the first dsRNA molecule and/or the second dsRNA molecule at, or near, the subcutaneous injection site. Without being bound by theory, the improved solubility of the formulation in the extracellular matrix may be due to the complexation of the first dsRNA molecule and/or the second dsRNA molecule with HPBCD, which may contribute to the increase in absorption rate.

In some embodiments, upon administration (e.g., subcutaneous administration) to subjects, the formulation exhibits a reduced relative variability of Tₘₐₓ across species (of subjects), as compared to a reference formulation (e.g., without HPBCD).

In some embodiments, upon administration (e.g., subcutaneous administration), the formulation exhibits a reduced relative variability of Tₘₐₓ between mice and monkeys, as compared to a reference formulation (e.g., without HPBCD).

In some embodiments, upon administration (e.g., subcutaneous administration), the formulation exhibits a reduced relative variability of Tₘₐₓ between rats and monkeys, as compared to a reference formulation (e.g., without HPBCD).

In some embodiments, upon administration (e.g., subcutaneous administration), the formulation exhibits a reduced relative variability of Tₘₐₓ between mice and humans, as compared to a reference formulation (e.g., without HPBCD).

In some embodiments, upon administration (e.g., subcutaneous administration), the formulation exhibits a reduced relative variability of Tₘₐₓ between rats and humans, as compared to a reference formulation (e.g., without HPBCD).

In some embodiments, upon administration (e.g., subcutaneous administration), the formulation exhibits a reduced relative variability of Tₘₐₓ between monkeys and humans, as compared to a reference formulation (e.g., without HPBCD).

In some embodiments, upon administration (e.g., subcutaneous administration) to a subject, the formulation exhibits an improved local tolerability profile due to reduced immunological and/or inflammatory response, as compared to a reference formulation (e.g., without HPBCD).

In some embodiments, the immunological and/or inflammatory response is determined by histopathologic analysis or measurement.

In some embodiments, the immunological and/or inflammatory response is determined by histopathologic analysis or measurement, wherein the analysis is determined by microscopic measurement.

In some embodiments, the immunological response is determined by a clinical scoring or grading system.

In some embodiments, the immunological response is determined by histopathologic analysis or measurement at the formulation injection site.

In some embodiments, the immunological response is determined by histopathologic analysis or measurement at the skin injection site.

In some embodiments, the immunological response is determined by histopathologic analysis or measurement at the skin injection site of immune cell presence or infiltration.

In some embodiments, the improved pharmacokinetic profile is determined by comparing the pharmacokinetic profiles of the formulation disclosed herein (e.g., comprising a first dsRNA molecule, a second dsRNA molecule, and a cyclodextrin agent), to a reference formulation (e.g., comprising a first dsRNA molecule and a second dsRNA molecule, but without a cyclodextrin agent).

In some embodiments, the improved pharmacokinetics result from the formulation disclosed herein (e.g., comprising a first dsRNA molecule, a second dsRNA molecule, and a cyclodextrin agent), wherein the improved pharmacokinetics is measured as a decrease or reduction in the time it takes to reach the maximum concentration (Tₘₐₓ; time to peak concentration) of the first dsRNA molecule and second dsRNA molecule upon administration, as compared to a reference formulation (e.g., comprising a first dsRNA molecule and a second dsRNA molecule, but without a cyclodextrin agent).

In some embodiments, the reduction in Tₘₐₓ is measured as a fold-change reduction in Tₘₐₓ of the formulation disclosed herein (e.g., comprising a first dsRNA molecule, a second dsRNA molecule, and a cyclodextrin agent) as compared to the Tₘₐₓ of a reference formulation (e.g., comprising a first dsRNA molecule and a second dsRNA molecule, but without a cyclodextrin agent), wherein the fold-change reduction in Tₘₐₓ is at least about 1.1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold. In some embodiments, the reduction in Tₘₐₓ is greater than 10-fold.

In some embodiments, the improved pharmacokinetics result from the formulation disclosed herein (e.g., comprising a first dsRNA molecule, a second dsRNA molecule, and a cyclodextrin agent), wherein the improved pharmacokinetics is indicated by the increase in the maximum concentration (Cₘₐₓ) of the first dsRNA molecule and second dsRNA molecule upon administration of the formulation disclosed herein, as compared to a reference formulation (e.g., comprising a first dsRNA molecule and a second dsRNA molecule, but without a cyclodextrin agent).

In some embodiments, the increase in Cₘₐₓ is measured as a fold-change increase in Cₘₐₓ of the formulation disclosed herein (e.g., comprising a first dsRNA molecule, a second dsRNA molecule, and a cyclodextrin agent) as compared to a reference formulation (e.g., comprising a first dsRNA molecule and a second dsRNA molecule, but without a cyclodextrin agent), wherein the fold change increase in Cₘₐₓ is at least 1.1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold. In some embodiments, the increase in Cₘₐₓ is greater than 10-fold.

In some embodiments, upon administration (e.g., subcutaneous administration) to a subject, the formulation (e.g., comprising a first dsRNA molecule, a second dsRNA molecule, and a cyclodextrin agent) exhibits a reduced Tₘₐₓ, and/or an increased Cₘₐₓ, as compared to a reference formulation (e.g., without HPBCD).

In some embodiments, upon administration (e.g., subcutaneous administration) to a subject, the formulation (e.g., comprising a first dsRNA molecule, a second dsRNA molecule, and a cyclodextrin agent) exhibits a reduced Tₘₐₓ as compared to a reference formulation (e.g., without HPBCD).

In some embodiments, upon administration (e.g., subcutaneous administration) to a subject, the formulation (e.g., comprising a first dsRNA molecule, a second dsRNA molecule, and a cyclodextrin agent) exhibits a reduced Tₘₐₓ by at least about 1.1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold, as compared to a reference formulation (e.g., without HPBCD).

In some embodiments, upon administration (e.g., subcutaneous administration) to a subject, the formulation (e.g., comprising a first dsRNA molecule, a second dsRNA molecule, and a cyclodextrin agent) exhibits an increased Cₘₐₓ, as compared to a reference formulation (e.g., without HPBCD).

In some embodiments, upon administration (e.g., subcutaneous administration) to a subject, the formulation (e.g., comprising a first dsRNA molecule, a second dsRNA molecule, and a cyclodextrin agent) exhibits an increased Cₘₐₓ by at least about 1.1-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold, as compared to a reference formulation (e.g., without HPBCD).

In some embodiments, the formulation (e.g., comprising a first dsRNA molecule, a second dsRNA molecule, and a cyclodextrin agent) enables the distribution of first and second dsRNA to tissue.

In some embodiments, the formulation (e.g., comprising a first dsRNA molecule, a second dsRNA molecule, and a cyclodextrin agent) enables the distribution of first and second dsRNA to liver, brain, heart, skin, lung, prostate, ovary, stomach, intestine, muscle, eye, throat, thymus, kidney, or placenta.

In some embodiments, the formulation (e.g., comprising a first dsRNA molecule, a second dsRNA molecule, and a cyclodextrin agent) enables the distribution of first and second dsRNA to the placenta.

In some embodiments, the formulation has a viscosity of about 50 cP or less, about 45 cP or less, about 40 cP or less, about 35 cP or less, about 30 cP or less, about 25 cP or less, about 20 cP or less, about 19 cP or less, about 18 cP or less, about 17 cP or less, about 16 cP or less, about 15 cP or less, about 14 cP or less, about 13 cP or less, about 12 cP or less, about 11 cP or less, or about 10 cP or less, as measured at about 25° C.

In some embodiments, the formulation has a viscosity of about 8 cP, about 9 cP, about 10 cP, about 11 cP, about 12 cP, about 13 cP, about 14 cP, about 15 cP, about 16 cP, about 17 cP, about 18 cP, about 19 cP, about 20 cP, as measured at about 25° C.

In some embodiments, a comparable or reference formulation, in the absence of the cyclodextrin agent, has a viscosity of greater than 20 cP, greater than 50 cP, greater than 100 cP, greater than 150 cP, greater than 200 cP, greater than 300 cP, greater than 500 cP, or even greater than 1,000 cP, as measured at about 25° C.

In some embodiments, the formulation has a viscosity being about 20% lower, about 30% lower, about 40% lower, about 50% lower, about 60% lower, about 70% lower, about 80% lower, about 90% lower, or more than about 90% lower, as compared to a comparable or reference formulation in the absence of the cyclodextrin agent, as measured at about 25° C.

In a preferred embodiment, the formulation contains a therapeutically effective amount of the dsRNA and has a volume of less than about 2 mL, less than about 1 mL, or less than about 0.75 mL.

In some embodiments, the formulation has a pH value ranging from about 6.5 to about 9.0.

In some embodiments, the formulation has a pH value of about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0.

In some embodiments, the formulation has a pH value ranging from about 6.0 to about 8.0.

In some embodiments, the formulation has a pH value ranging from about 6.5 to about 7.5.

In some embodiments, the formulation has a pH value of about 7.0±2.0, about 7.0±1.5, about 7.0±1.0, about 7.0±0.9, about 7.0±0.8, about 7.0±0.7, about 7.0±0.6, about 7.0±0.5, about 7.0±0.4, about 7.0±0.3, about 7.0±0.2, or about 7.0±0.1 (e.g., about 7.0).

In some embodiments, the pH value of the formulation is measured at about 1 day, about 2 days, about 3 days, about 4 days, about 5 days, about 6 days, about 7 days, about 14 days, about 20 days, about 30 days, about 60 days, about 90 days after the preparation of the formulation.

In some embodiments, the formulation has an osmolarity ranging from about 280 mOsm/L to about 310 mOsm/L.

In some embodiments, the formulation has an osmolarity of greater than about 250 mOsm/L, greater than about 300 mOsm/L, greater than about 350 mOsm/L, greater than about 400 mOsm/L, or greater than about 500 mOsm/L.

In some embodiments, the formulation has an osmolarity ranging from about 200 mOsm/L to about 2,000 mOsm/L, or from about 300 mOsm/L to about 1,000 mOsm/L.

### Exemplary Embodiments of Formulations

In some embodiments, the formulation comprises:
a dsRNA molecule, wherein the dsRNA molecule is present at a total concentration ranging from about 150 mg/mL to about 250 mg/mL; and
a cyclodextrin agent (e.g., HPBCD) present at a concentration ranging about 5% w/v to about 10% w/v, preferably a concentration of about 6.5% w/v.

In some embodiments, the formulation comprises:
a dsRNA molecule, wherein the dsRNA molecule is present at a total concentration ranging from about 150 mg/mL to about 250 mg/mL; and
a cyclodextrin agent (e.g., HPBCD) present in the formulation at a HPBCD:dsRNA molar ratio of no more than 5, preferably at a HPBCD:dsRNA molar ratio of between 2.5 and 5.

In some embodiments, the formulation comprises:
a first dsRNA molecule and a second dsRNA molecule, wherein first dsRNA molecule and the second dsRNA molecule are present at a total concentration ranging from about 150 mg/mL to about 250 mg/mL; and
a cyclodextrin agent (e.g., HPBCD) present at a concentration ranging about 5% w/v to about 10% w/v, preferably a concentration of about 6.5% w/v.

In some embodiments, the formulation comprises:
a first dsRNA molecule and a second dsRNA molecule, wherein first dsRNA molecule and the second dsRNA molecule are present at a total concentration ranging from about 150 mg/mL to about 250 mg/mL; and
a cyclodextrin agent (e.g., HPBCD) present in the formulation at a HPBCD:dsRNA molar ratio of no more than 5, preferably at a HPBCD:dsRNA molar ratio of between 2.5 and 5.

In some embodiments, the formulation comprises:
a first dsRNA molecule and a second dsRNA molecule, wherein first dsRNA molecule and the second dsRNA molecule are present at a total concentration ranging from about 150 mg/mL to about 250 mg/mL;
a cyclodextrin agent (e.g., HPBCD) present at a concentration ranging about 4% w/v to about 10% w/v, preferably a concentration of about 6.5% w/v; and
a buffering agent (e.g., sodium phosphate dibasic, potassium phosphate monobasic, or a combination thereof); and
a tonicity adjusting agent (e.g., sodium chloride, potassium chloride, or a combination thereof).

In some embodiments, the formulation comprises:
a first dsRNA molecule and a second dsRNA molecule, wherein first dsRNA molecule and the second dsRNA molecule are present at a total concentration ranging from about 150 mg/mL to about 250 mg/mL; and
a cyclodextrin agent (e.g., HPBCD) present at a concentration ranging about 4% w/v to about 10% w/v, preferably a concentration of about 6.5% w/v,
wherein the formulation has a physiologically suitable pH value, optionally, wherein the formulation has a pH value ranging from about 6.0 to about 8.0, from about 6.5 to about 7.5, or from about 7 to about 7.5.

In some embodiments, the formulation comprises:
a first dsRNA molecule and a second dsRNA molecule, wherein first dsRNA molecule and the second dsRNA molecule are present at a total concentration ranging from about 150 mg/mL to about 250 mg/mL; and
a cyclodextrin agent (e.g., HPBCD) present at a concentration ranging about 5% w/v to about 10% w/v, preferably a concentration of about 6.5% w/v,
wherein the formulation has a physiological osmolarity of greater than about 250 mOsm/L, greater than about 300 mOsm/L, greater than about 350 mOsm/L, greater than about 400 mOsm/L, or greater than about 500 mOsm/L.

In some embodiments, the formulation comprises:
a first dsRNA molecule and a second dsRNA molecule, wherein first dsRNA molecule and the second dsRNA molecule are present at a total concentration ranging from about 150 mg/mL to about 250 mg/mL; and
a cyclodextrin agent (e.g., HPBCD) present at a concentration ranging about 4% w/v to about 10% w/v, preferably a concentration of about 6.5% w/v,
wherein formulation has a physiologically suitable pH value, optionally, wherein the formulation has a pH value ranging from about 6.0 to about 8.0, from about 6.5 to about 7.5, or from about 7 to about 7.5, and
wherein the formulation has a physiological osmolarity of greater than about 250 mOsm/L, greater than about 300 mOsm/L, greater than about 350 mOsm/L, greater than about 400 mOsm/L, or greater than about 500 mOsm/L

In some embodiments, the formulation comprises one or more of the ingredients described in Table A.

TABLE A

| Ingredients | Concentration |
|---|---|
| First dsRNA | (total concentration) about 150 mg/L, about 155 mg/L, about 160 mg/mL, |
| Molecule and | about 165 mg/mL, about 170 mg/L, about 175 mg/L, about 180 mg/L, about |
| Second dsRNA | 185 mg/L, 190 mg/mL, 195 mg/mL, about 200 mg/L, about 205 mg/L, about |
| Molecule | 210 mg/L, about 215 mg/L, about 220 mg/L, about 225 mg/L, about 230 |
| | mg/L, about 235 mg/L, about 240 mg/L, about 245 mg/L, or about 250 mg/L |
| HPBCD | about 5.0% w/v, about 5.5% w/v, about 6.0% w/v, about 6.5% w/v, about |
| | 7.0% w/v, about 7.5% w/v, about 8% w/v, about 8.5% w/v, about 9% w/v, |
| | about 9.5% w/v, or about 10% w/v |

In some embodiments, the formulation comprises one or more of the ingredients described in Table B.

TABLE B

| Ingredients | Concentration |
|---|---|
| First dsRNA molecule (free acid) | about 87.5 mg/mL* |
| Second dsRNA molecule (free acid) | about 87.5 mg/mL* |
| HPBCD | From about 65 mg/mL to |
| | about 70 mg/mL |

*Added as a sodium salt. 1.06 mg of the sodium salt (anhydrous) is equivalent to 1 mg of free acid.

In some embodiments, the formulation further comprises one or more of the ingredients described in Table C.

TABLE C

| Ingredients | Concentration |
|---|---|
| Water | add to about 100% w/w |
| Sodium | about 0.50% w/v, about 0.45% w/v, about 0.40% w/v, about 0.35% w/v, |
| Chloride | about 0.30% w/v, about 0.25% w/v, about 0.20% w/v, about 0.15% w/v, or |
| | about 0.10% w/v |
| Potassium | about 0.50% w/v, about 0.45% w/v, about 0.40% w/v, about 0.35% w/v, |
| chloride | about 0.30% w/v, about 0.25% w/v, about 0.20% w/v, about 0.15% w/v, or |
| | about 0.10% w/v |
| Sodium | about 0.10% w/v, about 0.09% w/v, about 0.08% w/v, about 0.07% w/v, |
| phosphate | about 0.06% w/v, about 0.05% w/v, about 0.04% w/v, about 0.03% w/v, |
| dibasic | about 0.02% w/v, or about 0.01% w/v |
| Potassium | about 0.10% w/v, about 0.09% w/v, about 0.08% w/v, about 0.07% w/v, |
| phosphate | about 0.06% w/v, about 0.05% w/v, about 0.04% w/v, about 0.03% w/v, |
| monobasic | about 0.02% w/v, or about 0.01% w/v |

In some embodiments, the formulation comprises:
a first dsRNA molecule present at a concentration of about 87.5±30 mg/mL, about 87.5±25 mg/mL, about 87.5±20 mg/mL, about 87.5±15 mg/mL, about 87.5±10 mg/mL, about 87.5±5 mg/mL, about 87.5±4 mg/mL, about 87.5±3 mg/mL, about 87.5±2 mg/mL, or about 87.5±1 mg/mL (e.g., about 87.5 mg/mL);
a second dsRNA molecule present at a concentration of about 87.5±30 mg/mL, about 87.5±25 mg/mL, about 87.5±20 mg/mL, about 87.5±15 mg/mL, about 87.5±10 mg/mL, about 87.5±5 mg/mL, about 87.5±4 mg/mL, about 87.5±3 mg/mL, about 87.5±2 mg/mL, or about 87.5±1 mg/mL (e.g., about 87.5 mg/mL); and
a cyclodextrin agent (e.g., HPBCD) present at a concentration of about 65±30 mg/mL, about 65±25 mg/mL, about 65±20 mg/mL, about 65±15 mg/mL, about 65±10 mg/mL, about 65±5 mg/mL, about 65±4 mg/mL, about 65±3 mg/mL, about 65±2 mg/mL, or about 65±1 mg/mL (e.g., about 65 mg/mL).

### Processes of Preparing Formulations

In some embodiments, the present disclosure provides a process for preparing a preparing a formulation disclosed herein for injection (e.g. subcutaneous injection).

In some embodiments, the process comprises the step of (i) combining a cyclodextrin agent and water for injection to form a cyclodextrin solution. In some embodiments, the cyclodextrin agent is HPBCD.

In some embodiments, the process comprises the step of (ii) mixing a first dsRNA molecule and a second dsRNA molecule at a ratio of 1:1, thereby forming a mixture of the dsRNA molecules. In some embodiments, the first dsRNA molecule is at a concentration of about 100 mg/mL, about 105 mg/mL, about 110 mg/L, about 115 mg/mL, about 120 mg/L, about 125 mg/mL, about 130 mg/mL, about 135 mg/mL, about 140 mg/L, about 145 mg/L, about 150 mg/L, about 155 mg/L, about 160 mg/mL, about 165 mg/mL, about 170 mg/L, about 175 mg/L, about 180 mg/L, about 185 mg/L, about 190 mg/mL, about 195 mg/mL, about 200 mg/L, about 205 mg/L, about 210 mg/L, about 215 mg/L, about 220 mg/L, about 225 mg/L, about 230 mg/L, about 235 mg/L, about 240 mg/L, about 245 mg/L, or about 250 mg/L. In some embodiments, the first dsRNA molecule is at a concentration of about 190 mg/mL. In some embodiments, the second dsRNA molecule is at a concentration of about 100 mg/mL, about 105 mg/mL, about 110 mg/L, about 115 mg/mL, about 120 mg/L, about 125 mg/mL, about 130 mg/mL, about 135 mg/mL, about 140 mg/L, about 145 mg/L, about 150 mg/L, about 155 mg/L, about 160 mg/mL, about 165 mg/mL, about 170 mg/L, about 175 mg/L, about 180 mg/L, about 185 mg/L, about 190 mg/mL, about 195 mg/mL, about 200 mg/L, about 205 mg/L, about 210 mg/L, about 215 mg/L, about 220 mg/L, about 225 mg/L, about 230 mg/L, about 235 mg/L, about 240 mg/L, about 245 mg/L, or about 250 mg/L. In some embodiments, the second dsRNA molecule is at a concentration of about 190 mg/mL. In some embodiments, the mixture of the dsRNA molecules is at a total concentration of about 100 mg/mL, about 105 mg/mL, about 110 mg/L, about 115 mg/mL, about 120 mg/L, about 125 mg/mL, about 130 mg/mL, about 135 mg/mL, about 140 mg/L, about 145 mg/L, about 150 mg/L, about 155 mg/L, about 160 mg/mL, about 165 mg/mL, about 170 mg/L, about 175 mg/L, about 180 mg/L, about 185 mg/L, about 190 mg/mL, about 195 mg/mL, about 200 mg/L, about 205 mg/L, about 210 mg/L, about 215 mg/L, about 220 mg/L, about 225 mg/L, about 230 mg/L, about 235 mg/L, about 240 mg/L, about 245 mg/L, or about 250 mg/L. In some embodiments, the mixture of the dsRNA molecules is at a total concentration of about 190 mg/mL.

In some embodiments, the process comprises the step of (iii) adding the cyclodextrin solution to the mixture of the dsRNA molecules to form a bulk solution comprising the dsRNA molecules. In some embodiments, the bulk solution comprises the dsRNA molecules at a total concentration of about 100 mg/mL, about 105 mg/mL, about 110 mg/L, about 115 mg/mL, about 120 mg/L, about 125 mg/mL, about 130 mg/mL, about 135 mg/mL, about 140 mg/L, about 145 mg/L, about 150 mg/L, about 155 mg/L, about 160 mg/mL, about 165 mg/mL, about 170 mg/L, about 175 mg/L, about 180 mg/L, about 185 mg/L, about 190 mg/mL, about 195 mg/mL, about 200 mg/L, about 205 mg/L, about 210 mg/L, about 215 mg/L, about 220 mg/L, about 225 mg/L, about 230 mg/L, about 235 mg/L, about 240 mg/L, about 245 mg/L, or about 250 mg/L. In some embodiments, the bulk solution comprises the dsRNA molecules at a total concentration of about 175 mg/L. In some embodiments, the cyclodextrin agent is HPBCD. In some embodiments, HPBCD is present in the bulk solution at a concentration of about 5.0% w/v, about 5.5% w/v, about 6.0% w/v, about 6.5% w/v, about 7.0% w/v, about 7.5% w/v, about 8% w/v, about 8.5% w/v, about 9% w/v, about 9.5% w/v, or about 10% w/v. In some embodiments, HPBCD is present in the bulk solution at a concentration of about 6.5±3.0% w/v, about 6.5±2.5% w/v, about 6.5±2.0% w/v, about 6.5±1.0% w/v, about 6.5±0.9% w/v, about 6.5±0.8% w/v, about 6.5±0.7% w/v, about 6.5±0.6% w/v, about 6.5±0.5% w/v, about 6.5±0.4% w/v, about 6.5±0.3% w/v, about 6.5±0.2% w/v, or about 6.5±0.1% w/v. In some embodiments, HPBCD is present in the bulk solution at a concentration of about 6.5% w/v. In some embodiments, the bulk solution has a viscosity of about 50 cP or less, about 45 cP or less, about 40 cP or less, about 35 cP or less, about 30 cP or less, about 25 cP or less, about 20 cP or less, about 19 cP or less, about 18 cP or less, about 17 cP or less, about 16 cP or less, about 15 cP or less, about 14 cP or less, about 13 cP or less, about 12 cP or less, about 11 cP or less, or about 10 cP or less, as measured at about 25° C. In some embodiments, the bulk solution has a viscosity of about 8 cP, about 9 cP, about 10 cP, about 11 cP, about 12 cP, about 13 cP, about 14 cP, about 15 cP, about 16 cP, about 17 cP, about 18 cP, about 19 cP, about 20 cP, as measured at about 25° C.

In some embodiments, the process comprises the step of (iv) optionally, adjusting the pH by adding a buffering agent. In some embodiments, the bulk solution has a pH value of about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In some embodiments, the bulk solution has a pH value of about 7.0±2.0, about 7.0±1.5, about 7.0±1.0, about 7.0±0.9, about 7.0±0.8, about 7.0±0.7, about 7.0±0.6, about 7.0±0.5, about 7.0±0.4, about 7.0±0.3, about 7.0±0.2, or about 7.0±0.1 (e.g., about 7.0). In some embodiments, the buffering agent is selected from sodium phosphate dibasic, potassium phosphate monobasic, or any combination thereof.

In some embodiments, the process comprises the step of (v) reducing bioburden.

In some embodiments, the process comprises the step of (vi) transferring the bulk solution to a sterile holding vessel and performing sterile filtration of the bulk solution in the sterile holding vessel.

In some embodiments, the process comprises the step of (vii) filling sterile vials with the filtered bulk solution and applying sterile stoppers to securely seal the vials.

In some embodiments, the process comprises one or more steps of:
(i) combining HPBCD and water for injection to form an HPBCD solution;
(ii) mixing a first dsRNA molecule at a concentration of about 190 mg/mL and a second dsRNA molecule at a concentration of about 190 mg/mL at a ratio of 1:1, thereby forming a mixture of the dsRNA molecules of about 190 mg/mL;
(iii) adding the HPBCD solution to the mixture of the dsRNA molecules to form a bulk solution comprising the dsRNA molecules at a concentration of about 175 mg/mL, wherein the HPBCD is present in the bulk solution at a concentration of about 6.5% w/v;
(iv) optionally, adjusting the pH by adding the buffering agent;
(v) reducing bioburden;
(vi) transferring the bulk solution to a sterile holding vessel and performing sterile filtration of the bulk solution in the sterile holding vessel; and
(vii) filling sterile vials with the filtered bulk solution at a concentration of 175 mg/mL and applying sterile stoppers to securely seal the vials.

Toxicity and therapeutic efficacy of the formulations can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method disclosed herein, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical formulations can be included in a container, pack or dispenser together with optional instructions for administration.

### Methods of Use

In some aspects, the present disclosure provides a method of treating or preventing a disease, comprising administering to a subject in need thereof a formulation disclosed herein.

In some aspects, the present disclosure provides a formulation disclosed herein for use in treating or preventing a disease in a subject in need thereof.

In some aspects, the present disclosure provides a method of inhibiting secreted soluble fms-like tyrosine kinase-1 (sFLT1) in a subject, comprising administering to the subject a formulation disclosed herein.

In some aspects, the present disclosure provides a formulation disclosed herein for use in inhibiting sFLT1 in a subject.

In some embodiments, the subject is an animal.

In some embodiments, the subject is a mammal.

In some embodiments, the subject in need thereof is a human.

In some embodiments, the disease is associated with elevated expression or activity of sFLT1.

In some embodiments, the administration of the formulation results in an inhibition of sFLT1.

In some embodiments, the administration of the formulation results in a reduced activity of sFLT1.

In some embodiments, the present disclosure provides both prophylactic and therapeutic methods of treating a subject at risk of (or susceptible to) a disease or disorder caused, in whole or in part, by secreted Flt1 protein.

In some embodiments, the disease or disorder is a liver disease or disorder.

In some embodiments, the disease or disorder is a kidney disease or disorder.

In some embodiments, the disease or disorder is a placental disease or disorder.

In some embodiments, the disease or disorder is a pregnancy-related disease or disorder.

In some embodiments, the disease or disorder is a disorder associated with the expression of soluble Flt1 protein and in which amplified expression of the soluble Flt1 protein leads to clinical manifestations of pre-eclampsia (PE), postpartum PE, eclampsia and/or HELLP syndrome.

In some embodiments, the disease or disorder is PE.

In some embodiments, the disease or disorder is postpartum PE.

In some embodiments, the disease or disorder is eclampsia.

In some embodiments, the disease or disorder is HELLP syndrome.

In some embodiments, the present disclosure provides a method for preventing in a subject, a disease or disorder as described above, by administering to the subject in need thereof a formulation disclosed herein. Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

In some embodiments, the present disclosure provides a method of treating subjects therapeutically, i.e., alter onset of symptoms of the disease or disorder. In some embodiments, the modulatory method disclosed herein involves contacting a cell expressing a gain-of-function mutant with a formulation disclosed herein comprising a therapeutic agent (e.g., a RNAi agent or vector or transgene encoding same) that is specific for one or more target sequences within the gene (e.g., SEQ ID NOs: 1 or 2 or any combinations thereof), such that sequence specific interference with the gene is achieved.

With regards to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype,” or “drug response genotype”). Thus, another aspect of the present disclosure provides methods for tailoring an individual's prophylactic or therapeutic treatment with a formulation disclosed herein comprising either the target gene molecules or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

The formulation disclosed herein comprising a therapeutic agent can be tested in an appropriate animal model. For example, an RNAi agent (or expression vector or transgene encoding same) as described herein can be used in an animal model to determine the efficacy, toxicity, or side effects of treatment with said agent. Alternatively, The formulation disclosed herein comprising a therapeutic agent can be used in an animal model to determine the mechanism of action of such an agent. For example, The formulation disclosed herein comprising a therapeutic agent can be used in an animal model to determine the efficacy, toxicity, or side effects of treatment with such an agent. Alternatively, the formulation disclosed herein comprising a therapeutic agent can be used in an animal model to determine the mechanism of action of such an agent.

The formulation disclosed herein comprising an RNA silencing agent can be administered to any patient diagnosed as having or at risk for developing a pregnancy-, liver- and/or kidney-related disorder, such as PE and/or eclampsia. In one embodiment, the patient is diagnosed as having a PE and/or eclampsia, and the patient is otherwise in general good health. For example, the patient is not terminally ill, and the patient is likely to live at least 2, 3, 5 or more years following diagnosis. The patient can be treated immediately following diagnosis, or treatment can be delayed until the patient is experiencing more debilitating symptoms, such as two or more symptoms of PE or one or more symptoms of eclampsia. In some embodiments, the patient has not reached an advanced stage of the disease.

Delivery of the formulation disclosed herein comprising an RNA silencing agent directly to an organ (e.g., directly to the placenta, liver and/or kidneys) can be at a dosage that is effective to treat or prevent a liver-, kidney- or pregnancy-related disease or disorder, e.g., PE, postpartum PE, eclampsia and/or HELLP syndrome.

The concentration of the RNA silencing agent composition is an amount sufficient to be effective in treating or preventing a disorder or to regulate a physiological condition in humans. The concentration or amount of RNA silencing agent administered will depend on the parameters determined for the agent and the method of administration, e.g. nasal, buccal, or pulmonary.

The present disclosure pertains to uses of the above-described formulation comprising a therapeutic agents for prophylactic and/or therapeutic treatments as described Infra.

The route of delivery can be dependent on the disorder of the patient. In some embodiments, a subject diagnosed with PE, postpartum PE, eclampsia and/or HELLP syndrome can be administered an anti-sFlt1 RNA silencing agent disclosed herein by IV or SC administration. In addition to an RNA silencing agent disclosed herein, a patient can be administered a second therapy, e.g., a palliative therapy and/or disease-specific therapy. The secondary therapy can be, for example, symptomatic (e.g., for alleviating symptoms), protective (e.g., for slowing or halting disease progression), or restorative (e.g., for reversing the disease process). For the treatment of PE, postpartum PE, eclampsia and/or HELLP syndrome, for example, symptomatic therapies can further include the drugs Atenolol, Hydralazine, Labetalol, magnesium sulfate, Methyldopa, Nicardipine, Nifedipine, sodium nitroprusside and the like.

In general, an RNA silencing agent disclosed herein can be administered by any suitable method. As used herein, topical delivery can refer to the direct application of an RNA silencing agent to any surface of the body, including the eye, a mucous membrane, surfaces of a body cavity, or to any internal surface. Formulations for topical administration may include transdermal patches, ointments, lotions, creams, gels, drops, sprays, and liquids. Conventional pharmaceutical carriers, aqueous, powder or oily bases, thickeners and the like may be necessary or desirable. Topical administration can also be used as a means to selectively deliver the RNA silencing agent to the epidermis or dermis of a subject, or to specific strata thereof, or to an underlying tissue.

### Definitions

As used herein, the articles “a,” “an,” and “the” refer to one or to more than one (i.e., to at least one, or to one or more) of the grammatical object of the article. By way of example, “a nucleic acid molecule” means one nucleic acid molecule or one or more nucleic acid molecule.

As used herein, the term “w/v” stands for weight/volume and refers to the weight of a solute (usually in milligrams or grams) dissolved in a given volume of solution (usually in milliliters).

As used herein, the term “dsRNA” includes the dsRNA itself and a salt thereof. Suitable salts include a sodium salt and a potassium salt.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a formulation that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The term “oligonucleotide” refers to a short polymer of nucleotides and/or nucleotide analogs. The term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

An RNAi agent, e.g., an RNA silencing agent, having a strand which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

As used herein, the term “transgene” refers to any nucleic acid molecule, which is inserted by artifice into a cell, and becomes part of the genome of the organism that develops from the cell. Such a transgene may include a gene that is partly or entirely heterologous (i.e., foreign) to the transgenic organism, or may represent a gene homologous to an endogenous gene of the organism. The term “transgene” also means a nucleic acid molecule that includes one or more selected nucleic acid sequences, e.g., DNAs, that encode one or more engineered RNA precursors, to be expressed in a transgenic organism, e.g., animal, which is partly or entirely heterologous, i.e., foreign, to the transgenic animal, or homologous to an endogenous gene of the transgenic animal, but which is designed to be inserted into the animal's genome at a location which differs from that of the natural gene. A transgene includes one or more promoters and any other DNA, such as introns, necessary for expression of the selected nucleic acid sequence, all operably linked to the selected sequence, and may include an enhancer sequence.

A gene “involved” in a disease or disorder includes a gene, the normal or aberrant expression or function of which effects or causes the disease or disorder or at least one symptom of said disease or disorder.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target (sFLT1) and non-target (flFLT1) genes) can differ by one or more nucleotides, e.g., at an intronic region. In some embodiments, the target and non-target genes can differ by one or more polymorphisms (e.g., Single Nucleotide Polymorphisms or SNPs). In some embodiments, the target and non-target genes can share less than 100% sequence identity. In some embodiments, the non-target gene may be a homologue (e.g. an orthologue or paralogue) of the target gene.

The term “polymorphism” as used herein, refers to a variation (e.g., one or more deletions, insertions, or substitutions) in a gene sequence that is identified or detected when the same gene sequence from different sources or subjects (but from the same organism) are compared. For example, a polymorphism can be identified when the same gene sequence from different subjects are compared. Identification of such polymorphisms is routine in the art, the methodologies being similar to those used to detect, for example, breast cancer point mutations. Identification can be made, for example, from DNA extracted from a subject's lymphocytes, followed by amplification of polymorphic regions using specific primers to said polymorphic region. Alternatively, the polymorphism can be identified when two alleles of the same gene are compared. In particular embodiments, the polymorphism is a single nucleotide polymorphism (SNP).

A variation in sequence between two alleles of the same gene within an organism is referred to herein as an “allelic polymorphism.” In some embodiments, the allelic polymorphism corresponds to a SNP allele. For example, the allelic polymorphism may comprise a single nucleotide variation between the two alleles of a SNP. The polymorphism can be at a nucleotide within a coding region but, due to the degeneracy of the genetic code, no change in amino acid sequence is encoded. Alternatively, polymorphic sequences can encode a different amino acid at a particular position, but the change in the amino acid does not affect protein function. Polymorphic regions can also be found in non-encoding regions of the gene. In exemplary embodiments, the polymorphism is found in a coding region of the gene or in an untranslated region (e.g., a 5′ UTR or 3′ UTR) of the gene.

As used herein, the term “RNA silencing agent” refers to an RNA which is capable of inhibiting or “silencing” the expression of a target gene. In some embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, and dual-function oligonucleotides as well as precursors thereof. In one embodiment, the RNA silencing agent is capable of inducing RNA interference. In some embodiments, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, ²N-methylguanosine and ²° 2N,N-dimethylguanosine.

As used herein, the term “microRNA” (“miRNA”), also referred to in the art as “small temporal RNAs” (“stRNAs”), refers to a small (10-50 nucleotide) RNA which are genetically encoded (e.g., by viral, mammalian, or plant genomes) and are capable of directing or mediating RNA silencing. An “miRNA disorder” shall refer to a disease or disorder characterized by an aberrant expression or activity of an miRNA.

As used herein, the term “dual functional oligonucleotide” refers to a RNA silencing agent having the formula T-L-m, wherein T is an mRNA targeting moiety, L is a linking moiety, and m is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA). As used herein, the term “linking moiety” or “linking portion” refers to a domain, portion or region of the RNA-silencing agent which covalently joins or links the mRNA.

As used herein, the term “antisense strand” of an RNA silencing agent, e.g., an siRNA or RNA silencing agent, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g. certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portions of universal nucleotides typically comprise a nitrogen-containing aromatic heterocyclic moiety.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water, the solvate formed is a hydrate; and if the solvent is alcohol, the solvate formed is an alcoholate. Hydrates are formed by the combination of one or more molecules of water with one molecule of the substance in which the water retains its molecular state as H₂O.

It is to be understood that the present disclosure provides methods for the preparation of the formulations described herein. The present disclosure also provides detailed methods for preparation of various formulations of the present disclosure according to the following Examples.

It is to be understood that, unless otherwise stated, any description of a method of treatment includes use of the compounds to provide such treatment as is described herein, as well as use of the compounds to prepare a medicament to treat such condition. The treatment includes treatment of human or non-human animals including rodents and other disease models.

It is to be understood that, unless otherwise stated, any description of a method of prevention includes use of the compounds to provide such prevention as is described herein, as well as use of the compounds to prepare a medicament to prevent such condition. The prevention includes prevention in human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

As used herein, the term “preventing,” “prevent,” or “protecting against” describes reducing or eliminating the onset of the symptoms or complications of such disease, condition or disorder.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

As used herein, “viscosity” refers to the resistance of a substance (typically a liquid) to flow. Viscosity is related to the concept of shear force; it can be understood as the effect of different layers of the fluid exerting shearing force on each other, or on other surfaces, as they move against each other. There are several measures of viscosity. The units of viscosity are Ns/m, known as Pascal-seconds (Pa-s). Viscosity can be “kinematic” or “absolute”. Kinematic viscosity is a measure of the rate at which momentum is transferred through a fluid. It is measured in Stokes (St). The kinematic viscosity is a measure of the resistive flow of a fluid under the influence of gravity. When two fluids of equal volume and differing viscosity are placed in identical capillary viscometers and allowed to flow by gravity, the more viscous fluid takes longer than the less viscous fluid to flow through the capillary. If, for example, one fluid takes 200 seconds (s) to complete its flow and another fluid takes 400 s, the second fluid is called twice as viscous as the first on a kinematic viscosity scale. The dimension of kinematic viscosity is length/time. Commonly, kinematic viscosity is expressed in centiStokes (cSt). The SI unit of kinematic viscosity is mm/s, which is equal to 1 cSt. The “absolute viscosity,” sometimes called “dynamic viscosity” or “simple viscosity,” is the product of kinematic viscosity and fluid density. Absolute viscosity is expressed in units of centipoise (cP). The SI unit of absolute viscosity is the milliPascal-second (mPa-s), where 1 Cp=1 mPa-s. Viscosity may be measured by using, for example, a viscometer at a given shear rate or multiple shear rates. An “extrapolated zero-shear” viscosity can be determined by creating a best fit line of the four highest-shear points on a plot of absolute viscosity versus shear rate, and linearly extrapolating viscosity back to zero-shear. Alternatively, for a Newtonian fluid, viscosity can be determined by averaging viscosity values at multiple shear rates. Viscosity can also be measured using a microfluidic viscometer at single or multiple shear rates (also called flow rates), wherein absolute viscosity is derived from a change in pressure as a liquid flows through a channel. Viscosity equals shear stress over shear rate. Viscosities of fluids measured with microfluidic viscometers can, in some embodiments, be directly compared to extrapolated zero-shear viscosities, for example those extrapolated from viscosities measured at multiple shear rates using a cone and plate viscometer.

The term “concentrated” or “high-concentration”, as generally used herein, describes liquid formulations having a final concentration of nucleic acid greater than about 100 mg/mL, preferably greater than about 150 mg/mL, more preferably greater than about 200 mg/mL, or most preferably greater than about 250 mg/mL.

The term “injectability” or “syringeability,” as generally used herein, refers to the injection performance of a pharmaceutical formulation through a syringe equipped with an 18-32 gauge needle, optionally thin walled. Injectability depends upon factors such as pressure or force required for injection, evenness of flow, aspiration qualities, and freedom from clogging. Injectability of the liquid pharmaceutical formulations may be assessed by comparing the injection force of a reduced-viscosity formulation to a standard formulation without added cyclodextrin agents. The reduction in the injection force of the formulation containing a cyclodextrin agent reflects improved injectability of that formulation. The reduced viscosity formulations have improved injectability when the injection force is reduced by at least 10%, preferably by at least 30%, more preferably by at least 50%, and most preferably by at least 75% when compared to a standard formulation having the same concentration of polysaccharide or nucleic acid under otherwise the same conditions, except for replacement of the cyclodextrin agent with an appropriate buffer of about the same concentration. Alternatively, injectability of the liquid pharmaceutical formulations may be assessed by comparing the time required to inject the same volume, such as 0.5 mL, or more preferably about 1 mL, of different liquid polysaccharide or nucleic acid formulations when the syringe is depressed with the same force.

The term “osmolarity,” as generally used herein, refers to the total number of dissolved components per liter. Osmolarity is similar to molarity but includes the total number of moles of dissolved species in solution. An osmolarity of 1 Osm/L means there is 1 mole of dissolved components per L of solution. Some solutes, such as ionic solutes that dissociate in solution, will contribute more than 1 mole of dissolved components per mole of solute in the solution. For example, NaCl dissociates into Na⁺ and Cl⁻ in solution and thus provides 2 moles of dissolved components per 1 mole of dissolved NaCl in solution. Physiological osmolarity is typically in the range of about 280 mOsm/L to about 310 mOsm/L.

The term “tonicity,” as generally used herein, refers to the osmotic pressure gradient resulting from the separation of two solutions by a semi-permeable membrane. In particular, tonicity is used to describe the osmotic pressure created across a cell membrane when a cell is exposed to an external solution. Solutes that can cross the cellular membrane do not contribute to the final osmotic pressure gradient. Only those dissolved species that do not cross the cell membrane will contribute to osmotic pressure differences and thus tonicity.

As used herein, the term “administering” means subcutaneous (i.e., “SC,” “subQ,” or “SQ”) administration, oral administration, administration as a suppository, topical contact or administration, intravenous, parenteral, intraperitoneal, intramuscular, intraosseous, intralesional, intrathecal, intracranial, intranasal, epidural, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent, chemotherapeutic, or treatment for a neurodegenerative disease). The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In some embodiments, the formulations of the compositions of the present disclosure can be delivered by the use of liposomes which fuse with the cellular membrane or are endocytosed, e.g., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

As used herein, the term “HELLP syndrome” is well known in the art. HELLP syndrome is a life-threatening obstetric complication usually considered complication of preeclampsia. “HELLP” is an abbreviation of the three main features of the syndrome: Hemolysis, Elevated Liver enzymes, and Low Platelet count.

As used herein, the “reference formulation” refers to a formulation containing similar active ingredients as in the formulation being compared with, but not containing one or more components in the formulation being compared with. In some embodiments, the reference formulation comprises the first dsRNA molecule and the second dsRNA molecule of the formulation being compared with, but the reference formulation does not comprise the cyclodextrin agent (e.g., HPBCD). In some embodiments, in the reference formulation, the cyclodextrin agent (e.g., HPBCD) with another component (e.g., a buffering agent (e.g., PBS buffer)). The formulation comparison involves determining the relative change in, for example, pharmaceutical or biological activity (e.g., biological activity of agent), or pharmacokinetics (e.g., rate of absorption, maximum concentration, bioavailability, and the like), or pharmacodynamics of a first formulation comprising a biological agent or agents (e.g., siRNA or siRNAs), wherein the formulation comprises a cyclodextrin, compared to a second reference formulation comprising a biological agent or agents (e.g., siRNA or siRNAs), wherein the second reference formulation does not comprises a cyclodextrin. Alternatively, other properties of the first formulation and second reference formulation may be compared, such as physiochemical properties. The comparison may be the quantitative or qualitative relative change of, for example, the biological activity, pharmacokinetics, pharmacodynamics, and/or physicochemical properties between the first formulation and second reference formulation. The comparison may be measured, qualified, or determined by a relative fold change in a specified metric or property of the first formulation compared to the second reference formulation.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

It is understood that values presented in the examples are approximate values, and are subject to various experimental and instrumental variations.

### Example 1. Evaluation of Comparison Formulations and Exemplary Formulations

For comparison, formulations using various excipients, including water, hydrophobic acid salts, carboxylic acids, sugar alcohols, and amino acids, were prepared and evaluated. Table 2 summarizes the characteristic results of these formulations.

TABLE 2

| | 1:1 First dsRNA Molecule | | |
|---|---|---|---|
| | and Second dsRNA | | |
| | Molecule Total | Viscosity | Osmolality |
| Excipients | Concentration (mg/mL) | (cP) | (mOsm/Kg) |
| Water | 150 | 77.52 | 268 |
| Sorbitol (6.5%) | 150 | 99.09 | 818 |
| Camphor sulfonic | 150 | 100.08 | 773 |
| acid (0.2M) | | | |
| Methane sulfonic | 150 | 98.12 | 749 |
| acid (0.2M) | | | |
| Sodium lactate | 150 | 98.15 | — |
| (0.2M) | | | |
| Arginine (0.2M) | 150 | 93.30 | 595 |
| Lysine (0.2M) | 150 | 73.77 | 557 |
| Proline (0.2M) | 150 | 71.12 | 567 |

Exemplary formulations comprising HPBCD were compared with formulations using other excipients. Table 3 summarizes the results of the comparison.

TABLE 3

| | 1:1 First dsRNA | | |
|---|---|---|---|
| | Molecule and Second | | |
| | dsRNA Molecule Total | Viscosity | Osmolality |
| Excipients | Concentration (mg/mL) | (cP) | (mOsm/Kg) |
| Water | 160 | 43.74 | 258 |
| HPBCD (10%) | 160 | 10.22 | 439 |
| Camphor sulfonic acid (0.1M) + Arginine (0.1M) | 160 | 50.26 | 540 |
| Camphor sulfonic acid sodium salt (0.2M) | 160 | 44.57 | 772 |
| HPBCD (10% w/v) + 0.05M EDTA sodium salt | 160 | 11.66 | 634 |

Exemplary formulations comprising various concentrations of HPBCD and the dsRNA molecules were further evaluated. Tables 4-5 summarize the results of the evaluation.

TABLE 4

| | 1:1 First dsRNA Molecule | | |
|---|---|---|---|
| % w/v | and Second dsRNA Molecule | Viscosity | Osmolality |
| (HPBCD) | Total Concentration (mg/mL) | (Cp) | (mOsm/Kg) |
| 0 | 190 | 138.69 | 340 |
| 2.5 | 190 | 51.07 | |
| 5 | 190 | 20.8 | 340 |
| 5 | 190 | 14.7 | 389 |
| 10 | 190 | 16.1 | 500 |

TABLE 5

| | 1:1 First dsRNA Molecule | | |
|---|---|---|---|
| % w/v | and Second dsRNA Molecule | Viscosity | Osmolality |
| (HPBCD) | Total Concentration (mg/mL) | (Cp) | (mOsm/Kg) |
| 0 | 200 | 190.19 | 340 |
| 2 | 200 | 117.10 | Not Tested |
| 4 | 200 | 47.47 | 503 |
| 6 | 200 | 26.54 | 551 |
| 8 | 200 | 22.27 | 565 |
| 4 | 175 | 25.71 | 428 |
| 6 | 175 | 14.77 | 446 |

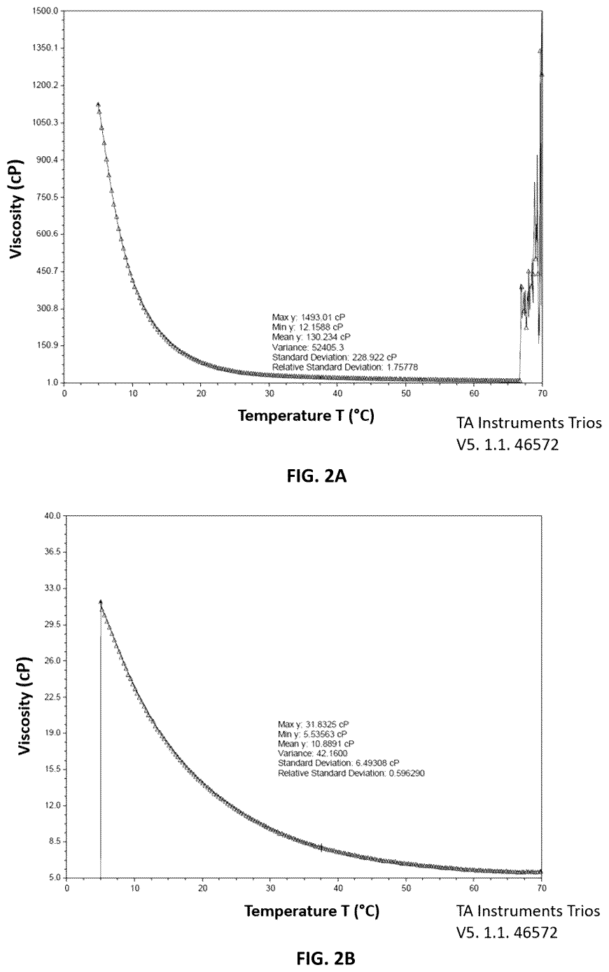

The data presented in FIGS. 2A and 2B reveal that the addition of 6.5% w/v of HPBCD resulted in a significant reduction in viscosity as well as lowered temperature-dependence of viscosity (i.e., viscosity was less sensitive to changes in temperature) in the formulation containing 1:1 ratio of the first and second dsRNA molecules at a total concentration of 150 mg/mL. This offers advantages during the manufacturing process and facilitates various manufacturing operations, such as mixing, pumping, and achieving uniform dispersion of the formulation.

Exemplary formulations comprising HPBCD with various degrees of HP substitution were further evaluated. Table 6 provides the results of the evaluation.

TABLE 6

| | 1:1 First dsRNA | | | |
|---|---|---|---|---|
| | Molecule and Second | | | |
| % w/v | dsRNA Molecule Total | Viscosity | Osmolality | |
| (HPBCD) | Concentration (mg/mL) | (Cp) | (mOsm/Kg) | pH |
| 0 | 175 | 75.07 | 300 | |
| 6.5 (MS)^{a} | 175 | 15.26 | 416 | 6.90 |
| 6.5 (HS)^{b} | 175 | 17.47 | 432 | |
| 6.5 (MS) | 175 | 14.52 | 424 | 6.86 |

^{a}Medium molar hydroxy propyl substitution. The nominal substitution value for HPBCD is 0.61.
^{b}High molar hydroxy propyl substitution. The nominal substitution value for HPBCD is 0.9.

Several formulations containing high concentrations of HPBCD (>10% w/v) in combination with dsRNA molecules were evaluated. Table 7 presents a comparison of the effects on viscosity between these high HPBCD concentrations (>10% w/v) and low HPBCD concentrations (10% w/v). The data in Table 7 suggests that the viscosity-lowering effect starts to decline when the molar ratio of HPBCD to dsRNA surpasses 5. The optimal range for the molar ratio of HPBCD to dsRNA, resulting in reduced viscosity, falls between 2.5 and 5. This range corresponds to a concentration of 5-10% of HPBCD for dsRNA concentrations exceeding 150 mg/mL.

TABLE 7

| | 1:1 dsRNA | | | |
|---|---|---|---|---|
| % w/v | Concentration | Molar Ratio | Viscosity | Osmolality |
| (HPBCD) | (mg/mL) | (HPBCD:dsRNA) | (Cp) | (mOsm/Kg) |
| 6.5 | 200 | 3.2 | 14.79 | 488 |
| 6.5 | 225 | 2.8 | 21.88 | 526 |
| 8 | 225 | 3.5 | 23.60 | 583 |
| 10 | 225 | 4.4 | 25.05 | 613 |
| 13 | 225 | 5.7 | 39.46 | Not Tested* |
| 20 | 225 | 8.7 | 59.58 | Not Tested* |
| 25 | 225 | 10.9 | 96.25 | Not Tested* |

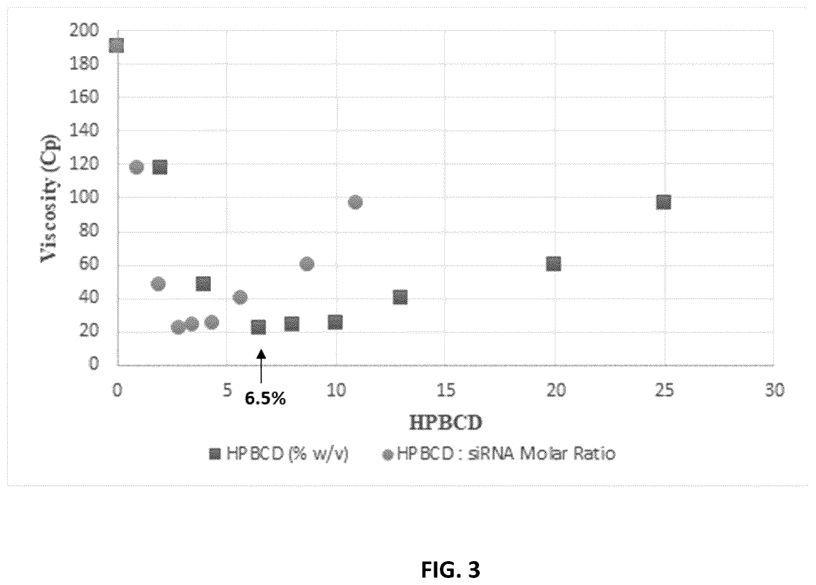

FIG. 3 further illustrates the effects of HPBCD concentration on the viscosity of dsRNA formulations. The reduction in viscosity by HPBCD was observed to be concentration-dependent, resulting in up to a 9-fold decrease when HPBCD concentration reached 6.5% w/v. However, an increase in viscosity was observed when HPBCD concentration exceeded 10% w/v. Without being bound by a theory, the increase in viscosity with HPBCD concentrations above 10% w/v could potentially be attributed to a shift in molecule interactions between HPBCD and dsRNA. Additionally, it was observed that the effectiveness of viscosity reduction declined when the molar ratio of HPBCD to dsRNA exceeded 5. The optimal range for HPBCD to dsRNA molar ratio for maximum viscosity reduction was found to be between 2.5 and 5. This range corresponded to a concentration of 5-10% w/v of HPBCD for dsRNA concentrations greater than 150 mg/mL. Moreover, for the formulation containing a total concentration of 225 mg/mL of the first and second dsRNA molecules at a 1:1 ratio, it was observed that the addition of HPBCD at a concentration of 6.5% w/v yielded the best results for optimal viscosity reduction.

For further evaluation, an exemplary formulation was prepared according to Table 8. The pH of the formulation was observed to be close to 7, indicating a neutral pH environment.

TABLE 8

| | Concentration | | Quality |
|---|---|---|---|
| Component | (Per mL) | | Standard |
| First dsRNA Molecule (free acid) | 87.5 | mg^{a} | In-House |
| Second dsRNA Molecule (free acid) | 87.5 | mg^{a} | In-House |
| Hydroxypropyl Beta Cyclodextrin | 65-70 | mg | USP |

| | | |
|---|---|---|
| Water for injection | Qs. to 1 mL | USP |

^{a}Added as a sodium salt. 1.06 mg of the sodium salt (anhydrous) is equivalent to 1 mg of free acid.

### Example 2. Comparative UV Absorptivity Analysis of HPBCD and Water for siRNA

### Formulation

In the study comparing HPBCD and water regarding UV absorptivity for the formulation with the first siRNA, second siRNA, and the combination of the two, the objective was to investigate if there were any differences in the UV absorptivity patterns between the two mediums. UV absorptivity serves as an important indicator of concentration-dependent response and can provide insights into the solubility and stability of molecules within a formulation.

The UV absorptivity analysis was conducted by measuring the absorbance spectra of the formulations containing the first siRNA, second siRNA, and the combination of both siRNAs in both HPBCD and water. The measurements were performed across a suitable range of wavelengths, allowing for a comprehensive evaluation of the UV absorbance patterns.

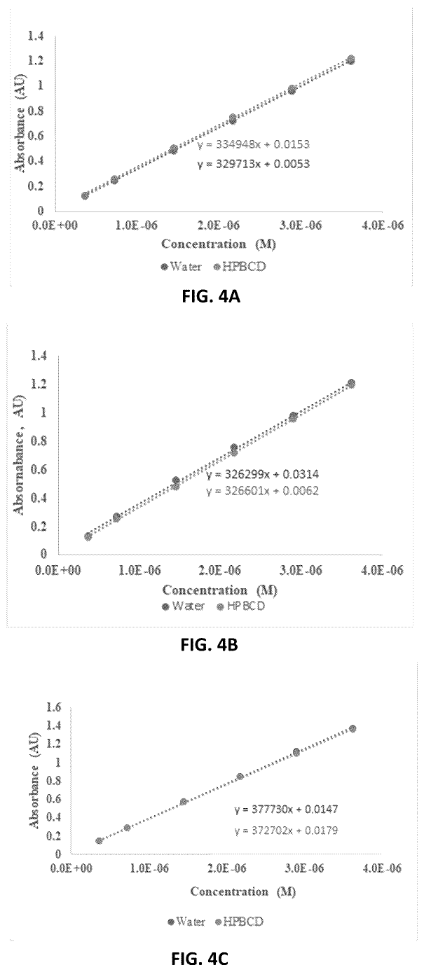

The results of the study revealed that HPBCD and water displayed the same absorptivity pattern, which was concentration-dependent. FIGS. 4A-4C show that the concentration of the siRNAs increased, the UV absorbance also increased accordingly. The UV absorbance spectra exhibited similar trends in both mediums for all three formulations.

The observed similarity in the UV absorptivity patterns between water and HPBCD suggests that HPBCD solution can be viable option as the medium for these siRNA formulations with no impact on analytical quantitation.

### Example 3. Exemplary Preparation of Formulations

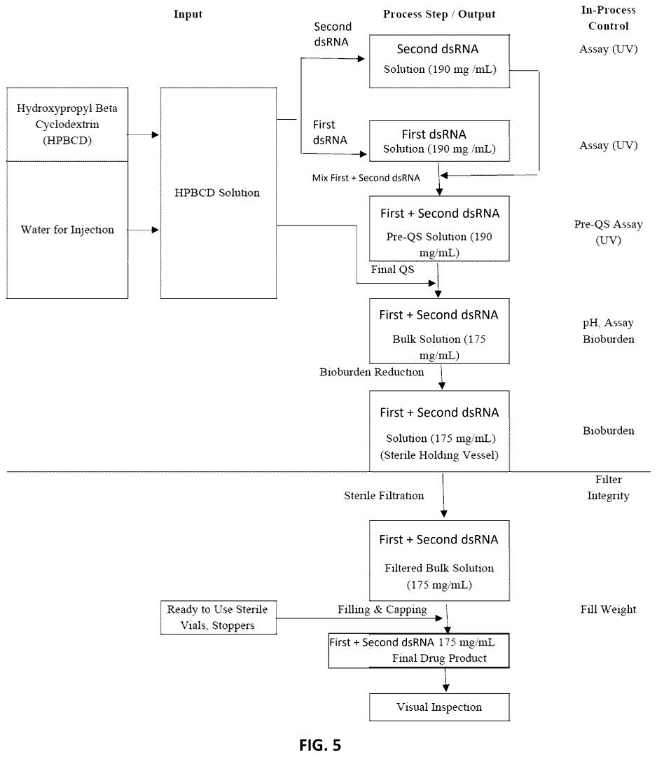

FIG. 5 depicts an exemplary preparation process of the dsRNA formulation.

The formulation preparation process begins with the combination of hydroxypropyl beta cyclodextrin (HPBCD) and water for injection, resulting in the formation of the HPBCD solution. This step ensures the proper solubility and availability of HPBCD for subsequent stages. Simultaneously, the first dsRNA molecule at a concentration of 190 mg/mL and the second dsRNA molecule at the same concentration are combined at the 1:1 ratio.

Once the first and second dsRNA molecules are combined, a PreQS solution is prepared at a concentration of 190 mg/mL. This solution contains the active agents and serves as a precursor for the subsequent steps. The HPBCD solution is then added to the container containing the active agents, resulting in the formation of a bulk solution. The bulk solution achieves a concentration of 175 mg/mL.

To ensure appropriate quality and stability, the bulk solution undergoes pH adjustment and bioburden reduction. The pH adjustment process is performed according to defined specifications, while bioburden reduction techniques are implemented to reduce microbial contamination risks. These measures contribute to the overall safety and efficacy of the formulation.

Following pH adjustment and bioburden reduction, the active agent solution is transferred to a sterile holding vessel. The sterile holding vessel maintains the integrity and sterility of the solution throughout subsequent processing steps.

The next stage involves sterile filtration, where the active agent solution within the sterile holding vessel undergoes filtration using a suitable sterile filtration system. This filtration process effectively removes any potential contaminants, ensuring the final product's purity and sterility.

After filtration, the filtered bulk solution, at a concentration of 175 mg/mL, is ready for the filling and capping stage. The filtered solution is dispensed into sterile vials. Once filled, the vials are securely sealed with sterile stoppers, ensuring the preservation of the formulation's integrity.

To guarantee the quality of the final product, visual inspection is performed on the filled and capped vials. This inspection aims to identify any visible defects such as particles, discoloration, or abnormalities that may compromise the product's quality or appearance.

Finally, the inspected and approved vials are stored in sterile conditions, adhering to specific temperature and environmental requirements to maintain stability and sterility until further use or distribution.

### Example 4. Pharmacokinetics of Formulations Following Intravenous or Subcutaneous Single Dose Administration

The pharmacokinetics and tissue distribution profiles of a formulation comprising the first dsRNA and the second dsRNA combined into a 1:1 ratio in HPBCD and PBS buffer were evaluated in non-pregnant female Sprague Dawley rats following a single intravenous (IV) dose administration. In addition, the pharmacokinetics and tissue distribution profiles the first dsRNA and the second dsRNA in HPBCD and PBS buffer were evaluated in non-pregnant female Sprague Dawley rats following a single subcutaneous (SC) dose administration.

### Intravenous Pharmacokinetics (Female Non-Pregnant Rats)

Plasma exposures (Cₘₐₓ and AUC_{inf}), concentrations (ng siRNA/g of tissue) and % recovery in liver and kidney following 5 mg/kg IV administration of each the first dsRNA and the second dsRNA in a 1:1 concentration ratio to an individual female non-pregnant rat, were comparable between HPBCD and PBS formulations as shown in Error! Reference source not found. and Error! Reference source not found., respectively. These results demonstrate that HPBCD does not impact overall pharmacokinetics of both siRNAs (i.e., the first dsRNA and the second dsRNA), as compared to the PBS control.

TABLE 9

| | | | | | | AUC_{INF}_ |
|---|---|---|---|---|---|---|
| | | | | | | obs |
| | Dose^{a} | | Cₘₐₓ | Tₘₐₓ | Half | ((h*ng/ |
| Formulation | (mg/kg) | Analyte | (ng/mL) | (h) | Life (h) | ml) |
| PBS Buffer | 5 | First | 127000 | 0.083 | 1.0 | 116000 |
| 6.5% w/v | 5 | dsRNA | 98100 | 0.083 | 1.1 | 107000 |
| HPBCD | | | | | | |
| PBS Buffer | 5 | Second | 113000 | 0.083 | 1.0 | 87700 |
| 6.5% w/v | 5 | dsRNA | 103000 | 0.083 | 1.0 | 78300 |
| HPBCD | | | | | | |

^{a}Dose of each siRNA was 5 mg/kg. Total dose of combination of the first and second dsRNA is 10 mg/kg

TABLE 10

| | | | | Average | | | Average |
|---|---|---|---|---|---|---|---|
| | | Liver (ng/g) | | Recovery | Kidney (ng/g) | | Recovery |
| | Dose | First | Second | (%) Both | First | Second | (%) Both |
| Formulation | (mg/kg)^{a} | dsRNA | dsRNA | siRNA's | dsRNA | dsRNA | siRNA's |
| PBS | 5.0 | 68800 | 73500 | 62.5% | 29400 | 22600 | 4.1% |
| Buffer | | | | | | | |
| 6.5% | 5.0 | 75200 | 76400 | 63.1% | 44100 | 28000 | 5.6% |
| HPBCD | | | | | | | |

^{a}Dose of each siRNA was 5 mg/kg. Total dose of combination of the first and second dsRNA is 10 mg/kg

### Subcutaneous Pharmacokinetics in Female Non-Pregnant Rats

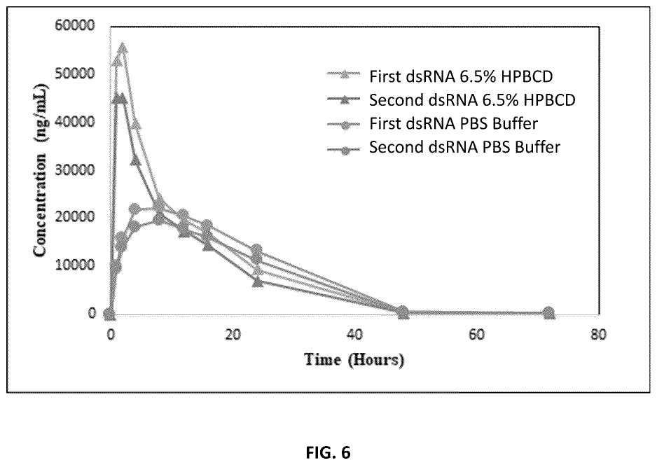

Plasma concentration profiles following 50 mg/kg subcutaneous (SC) administration of each siRNA (i.e., the first dsRNA and the second dsRNA in 1:1 ratio) in HPBCD and PBS formulations are shown in FIG. 6. The HPBCD formulation demonstrated a lower Tₘₐₓ and greater than 2-fold higher Cₘₐₓ for both the first dsRNA and the second dsRNA, respectively, when compared with formulation of the first dsRNA and the second dsRNA in PBS buffer. These results are consistent with an increase in absorption rate for the HPBCD formulation than that for the PBS buffer formulations.

Plasma (AUCₗₐₛₜ) exposures following 50 mg/kg SC administration of each of the first and second dsRNA were comparable between the 6.5% (w/v) and 8% (w/v) HPBCD and PBS formulations as shown in Table 11. Concentrations (ng siRNA/g of tissue) and % recovery of both siRNAs in liver and kidney at 72 hours are similar between the 6.5% (w/v) and 8% (w/v) HPBCD and PBS formulations as shown in Table 12. Accordingly, HPBCD formulation provided rapid absorption from the injection site with no impact on overall exposure and final tissue distribution.

TABLE 11

| | Dose^{a} | | | Cₘₐₓ | Tₘₐₓ | Half | AUCₗₐₛₜ |
|---|---|---|---|---|---|---|---|
| Formulation | (mg/kg) | Analyte | N = 3 | (ng/mL) | (h) | Life (h) | (h*ng/ml) |
| PBS Buffer | 50 | First | Mean | 23400 | 8 | 7.7 | 609000 |
| 6.5% w/v | | dsRNA | Mean | 56800 | 1 | 8.9 | 690000 |
| HPBCD | | | | | | | |
| 8% w/v | | | Mean | 57700 | 2 | 8.6 | 684000 |
| HPBCD | | | | | | | |
| PBS Buffer | 50 | Second | Mean | 19900 | 8 | 7.8 | 518000 |
| 6.5% w/v | | dsRNA | Mean | 47500 | 1 | 8.8 | 567000 |
| HPBCD | | | | | | | |
| 8% w/v | | | Mean | 49800 | 2 | 8.1 | 594000 |
| HPBCD | | | | | | | |

^{a}Dose of each siRNA was 50 mg/kg. Total dose of combination of the first and second dsRNA is 100 mg/kg

TABLE 12

| | | | | Average | | | Average |
|---|---|---|---|---|---|---|---|
| | | | | Recovery | | | Recovery |
| | | Liver (ng/g) | | (%) | Kidney (ng/g) | | (%) |
| | Dose^{a} | First | First | Both | First | First | Both |
| Formulation | (mg/kg) | dsRNA | dsRNA | SIRNA's | dsRNA | dsRNA | SIRNA's |
| PBS Buffer | 50 | 603000 | 638000 | 57.80% | 2600000 | 2050000 | 37.60% |
| 6.5% w/v | | 750500 | 728000 | 63.10% | 1780000 | 1330000 | 23.20% |
| HPBCD | | | | | | | |
| 8% w/v | | 638000 | 696000 | 53.60% | 2080000 | 1730000 | 28.85% |
| HPBCD | | | | | | | |

^{a}Dose of each siRNA was 50 mg/kg. Total dose of combination of the first and second dsRNA is 100 mg/kg

### Subcutaneous Pharmacokinetics in Pregnant Rats

Pharmacokinetic parameters following 35 mg/kg or 100 mg/kg SC administration of the first dsRNA and the second dsRNA in HPBCD and PBS formulations, respectively, are shown in Table 13. The HPBCD formulation demonstrated a lower Tₘₐₓ and higher Cₘₐₓ for both siRNAs when compared with formulations in PBS buffer alone. These results are consistent with an increase in absorption rate for the HPBCD formulation than that for the PBS buffer formulations. Plasma (AUₗₐₛₜ) exposures following 35 mg/kg or 100 mg/kg SC administration of each of the first dsRNA and the second dsRNA were comparable between HPBCD and PBS formulations, as shown in Table 13.

TABLE 13

| | Dose^{a} | | Cₘₐₓ | Tₘₐₓ | Half | AUCₗₐₛₜ |
|---|---|---|---|---|---|---|
| Formulation | (mg/kg) | Analyte | (ng/ml) | (h) | Life (h) | (h*ng/ml) |
| PBS Buffer | 35 | First | 16500 | 8 | 4.8 | 287000 |
| 6.5% w/v HPBCD | | dsRNA | 26200 | 2 | 5.4 | 298000 |
| PBS Buffer | 100 | First | 67100 | 8 | 8.1 | 1230000 |
| 6.5% w/v HPBCD | | dsRNA | 88800 | 4 | 8.6 | 1310000 |
| PBS Buffer | 35 | Second | 15000 | 8 | 4.8 | 255000 |
| 6.5% w/v HPBCD | | dsRNA | 22800 | 2 | 5.9 | 269000 |
| PBS Buffer | 100 | Second | 53900 | 8 | 8.4 | 982000 |
| 6.5% w/v HPBCD | | dsRNA | 69600 | 4 | 8.7 | 1070000 |

^{a}Dose of each siRNA was either 35 mg/kg or 100 mg/kg. Total dose of combination of the first and second dsRNA is 70 mg/kg or 200 mg/kg.

Concentrations (ng siRNA/g of tissue) and % recovery of both siRNAs in liver, kidney and placenta at 72 hours are similar between the two formulations, as shown in Table 14. Accordingly, HPBCD formulation provided rapid absorption from the injection site with no impact on overall exposure and final tissue distribution.

TABLE 14

| | | | | Average | | | Average | | | Average |
|---|---|---|---|---|---|---|---|---|---|---|
| | | | | Recovery | | | Recovery | Placenta | | Recovery |
| | Dose^{a} | Liver (ng/g) | | (%) | Kidney (ng/g) | | (%) | (ng/g) | | (%) |
| | (mg/ | First | Second | Both | First | Second | Both | First | Second | Both |
| Formulation | kg) | dsRNA | dsRNA | siRNAs | dsRNA | dsRNA | siRNAs | dsRNA | dsRNA | siRNAs |
| PBS | 35 | 254000 | 285000 | 50.5% | 556000 | 420000 | 11.2% | 14400 | 12600 | 0.9% |
| Buffer | | | | | | | | | | |
| 6.5% w/v | | 273000 | 323000 | 48.0% | 437000 | 363000 | 8.5% | 14800 | 13300 | 0.9% |
| HPBCD | | | | | | | | | | |
| PBS | 100 | 383000 | 444000 | 26.0% | 2290000 | 2080000 | 17.6% | 70700 | 62300 | 1.9% |
| Buffer | | | | | | | | | | |
| 6.5% w/v | | 444000 | 513000 | 29.7% | 1970000 | 1970000 | 15.2% | 58700 | 52800 | 1.6% |
| HPBCD | | | | | | | | | | |

^{a}Dose of each siRNA was either 35 mg/kg or 100 mg/kg. Total dose of combination of the first and second dsRNA is 70 mg/kg or 200 mg/kg.

### Assessment of Subcutaneous Injection Site Reactions in Pregnant Rats

Local tolerability and inflammatory response at the injection site was assessed following subcutaneous injection of a formulation either comprising a 1:1 mixture of the first and second dsRNA and HPBCD (i.e., Formulation 2 in Table 15) or a formulation comprising a 1:1 mixture of the first and second dsRNA in PBS (i.e., Formulation 1 in Table 15).

Histopathologic findings for the injection site were initially graded from one to five, depending upon severity (1 being least severe and 5 being most severe) were determined for 6 animals per dose group. The results primarily indicate minimal to mild mononuclear cell infiltrates involving the dermis and/or subcutaneous tissue across animal Groups 2, 3, 4, and 5, and mild to moderate mononuclear cell inflammation involving the dermis and/or subcutaneous tissue across animal Groups 2, 4, and 5.

Moreover, the most notable difference across the animal dose Groups was the absence and reduced inflammatory reaction at 70 mg/kg dose, as well as the higher 200 mg/kg dose, using the 6.5% w/v HPBCD (Formulation 2) which could be attributed to the observed faster absorption rate from the injection site.

TABLE 15

| | | Group Number/Dose Route | | | |
|---|---|---|---|---|---|
| | | 2 | 3 | 4 | 5 |
| | | (SC-1) | (SC-2) | (SC-3) | (SC-4) |
| | | Dose (mg/kg) | | | |
| | | 70 | 70 | 200 | 200 |
| | | Dose Volume (mL/kg) | | | |
| | | 0.70 | 0.40 | 2.00 | 1.14 |
| | | Formulation^{a} | | | |
| | | 1 | 2 | 1 | 2 |
| | | Formulation Concentration | | | |
| | | 100 | 175 | 100 | 175 |
| | | mg/mL | mg/mL | mg/mL | mg/mL |
| | | Number of Animals Per Dose Group | | | |
| | | 6 | 6 | 6 | 6 |
| SKIN, | Number | 6 | 6 | 6 | 6 |
| INJECTION | Examined | | | | |
| SITE | Unremarkable | 1 | 1 | 0 | 0 |
| DERMIS | | | | | |
| Infiltrate, | minimal | 0 | 1 | 0 | 0 |
| mononuclear cell | | | | | |
| DERMIS/SUBCUTANEOUS | | | | | |
| Infiltrate, | minimal | 0 | 1 | 0 | 0 |
| mononuclear cell | mild | 0 | 1 | 0 | 0 |
| Inflammation, | mild | 0 | 0 | 1 | 0 |
| mononuclear cell | moderate | 2 | 0 | 1 | 1 |
| EPIDERMAL | | | | | |
| Hyperplasia | minimal | 1 | 3 | 3 | 3 |
| | mild | 0 | 0 | 1 | 0 |
| SUBCUTANEOUS | | | | | |
| Infiltrate, | minimal | 1 | 2 | 0 | 2 |
| mononuclear cell | mild | 0 | 0 | 2 | 1 |
| Inflammation, | mild | 0 | 0 | 0 | 1 |
| mononuclear cell | moderate | 2 | 0 | 2 | 1 |

SC: Subcutaneous
^{a}Formulation 1 is a 1:1 mixture of first dsRNA and second dsRNA at 100 mg/mL in 0.4% PBS; Formulation 2 is a 1:1 mixture of first dsRNA and second dsRNA at 175 mg/mL in 6.5% HPBCD.

The composition of Formulation 2 as tested in Table 15 is provided in Table 16.

TABLE 16

| | | Concentration | | | | Quality |
|---|---|---|---|---|---|---|
| Component | Function | (Per mL) | | Per vial^{b} | | Standard |
| First dsRNA | Active | 87.5 | mg^{a} | 78.8 | mg^{b} | In-House |
| (free acid) | Ingredient | | | | | |
| Second dsRNA | Active | 87.5 | mg^{a} | 78.8 | mg^{b} | In-House |
| (free acid) | Ingredient | | | | | |
| Hydroxypropyl | Viscosity | 65 | mg^{c} | 58.5 | mg^{b} | USP |
| Beta Cyclodextrin | Modifier | | | | | |

| | | | | |
|---|---|---|---|---|
| Water for | Solvent | Qs. to 1 mL | Qs^{d} | USP |
| injection | | | | |

^{a}Added as a sodium salt. 1.06 mg of the sodium salt (anhydrous) is equivalent to 1 mg of free acid. Adjusted as needed based on the potency of each of the drug substance lots used in the manufacture of the drug product
^{b}Based on a labelled volume of 0.9 mL. An overfill following USP <1151> is applied to ensure the labelled content is delivered
^{c}On an anhydrous basis. Corrected for water content
^{d}Quantity sufficient to provide 87.5 mg/mL concentration of each of the drug substance and a total concentration of 175 mg/mL
USP = United States Pharmacopoeia

### EQUIVALENTS

It is to be understood that the invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1-94. (canceled)

95. A formulation comprising:
(i) a dsRNA molecule; and
(ii) a cyclodextrin agent,
wherein the cyclodextrin agent is present in the formulation at a cyclodextrin:dsRNA molar ratio of no more than 5.

96. The formulation of claim 95, wherein the dsRNA molecule is present at a concentration of at least about 50 mg/mL, at least about 100 mg/mL, at least about 150 mg/mL, or about 150 mg/mL to 250 mg/mL.

97. A formulation comprising:
(i) a first dsRNA molecule and a second dsRNA molecule; and
(ii) a cyclodextrin agent.

98. The formulation of claim 97, wherein the first dsRNA molecule and the second dsRNA molecule are present at a total concentration of at least about 50 mg/mL, at least about 100 mg/mL, at least about 150 mg/mL, or about 150 mg/mL to 250 mg/mL.

99. The formulation of claim 95, wherein the cyclodextrin agent is present at a concentration ranging from about 5% w/v to about 35% w/v, about 5% w/v to about 25% w/v, or about 5% w/v to about 10% w/v.

100. The formulation of claim 95, wherein the formulation has a viscosity of about 8 cP, about 9 cP, about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, about 23 cP, about 24 cP, or about 25 cP, as measured at about 25° C.

101. The formulation of claim 95, wherein the dsRNA molecule is present at a concentration of at least about 150 mg/mL, and the cyclodextrin agent is present at a concentration ranging about 5% w/v to about 35% w/v.

102. The formulation of claim 95, wherein the dsRNA molecule is present at a concentration ranging from about 150 mg/mL to about 250 mg/mL.

103. The formulation of claim 95, wherein the cyclodextrin agent is hydroxypropyl 3-cyclodextrin (HPBCD).

104. The formulation of claim 95, comprising one or more of a buffering agent, a tonicity adjusting agent, and a physiologically suitable pH value.

105. The formulation of claim 95, wherein the formulation has a physiological osmolarity of greater than about 250 mOsm/L, greater than about 300 mOsm/L, greater than about 350 mOsm/L, greater than about 400 mOsm/L, or greater than about 500 mOsm/L.

106. A formulation comprising:
(i) a dsRNA molecule; and
(ii) a cyclodextrin agent,
wherein the dsRNA molecule comprises an antisense strand and a sense strand, each strand with a 5′ end and a 3′ end, wherein:
(1) the antisense strand comprises a sequence substantially complementary to a nucleic acid sequence of 5′ CTCTCGGATCTCCAAATTTA 3′ (SEQ ID NO:1) or 5′ CATCATAGCTACCATTTATT 3′ (SEQ ID NO:2);
(2) the antisense strand is at least 20 nucleotides in length;
(3) the antisense strand comprises at least 50% 2′-O-methyl modifications;
(4) the nucleotides at any one or more of positions 2, 4, 5, 6, 8, 10, 12, 14, 16, and 20 from the 5′ end of the antisense strand are not 2′-methoxy-ribonucleotides;
(5) the nucleotides at positions 1-2 to 1-8 from the 3′ end of the antisense strand are connected to each other via phosphorothioate internucleotide linkages;
(6) a portion of the antisense strand is complementary to a portion of the sense strand;
(7) the sense strand is at least 15 nucleotides in length;
(8) the sense strand comprises at least 65% 2′-O-methyl modifications;
(9) the nucleotides at any one of more of positions 4, 6, 8, 10, and 14 from the 5′ end of the sense strand are not 2′-methoxy-ribonucleotides; and
(10) the nucleotides at positions 1-2 from the 5′ end of the sense strand are connected to each other via phosphorothioate internucleotide linkages.

107. The formulation of claim 106, wherein the antisense strand is 20 nucleotides, 21 nucleotides, or 22 nucleotides in length; and/or the sense strand is 15 nucleotides, 16 nucleotides, 18, or 20 nucleotides in length.

108. The formulation of claim 106, wherein the dsRNA molecule comprises a double-stranded region of 15 base pairs to 20 base pairs.

109. The formulation of claim 106, wherein the dsRNA molecule comprises a blunt-end or at least one single stranded nucleotide overhang.

110. The formulation of claim 106, wherein the antisense strand comprises a 5′ vinyl phosphonate.

111. The formulation of claim 106, wherein a functional moiety is linked to the 3′ end of the sense strand.

112. The formulation of claim 111, wherein the functional moiety:
(i) comprises a hydrophobic moiety and/or
(ii) is linked to the sense strand by a cleavable linker.

113. The formulation of claim 106, wherein the nucleotides at positions 1 and 2 from the 3′ end of sense strand, and the nucleotides at positions 1 and 2 from the 5′ end of antisense strand, are connected to adjacent ribonucleotides via phosphorothioate linkages.

114. A method of treating or preventing a disease, comprising administering to a subject in need thereof a formulation comprising:
(i) a dsRNA molecule; and
(ii) a cyclodextrin agent,
wherein the cyclodextrin agent is present in the formulation at a cyclodextrin:dsRNA molar ratio of no more than 5.
